SEQUENCE LISTING (20 sequences)

SEQ ID NO: 1 - DNA, 612 bp, Pseudomonas putida
atgagccccaagaggagaacccaggccgagagagccatggagacccagggcaagctgatcgccgctgccctgggcgtgctgagagagaagggctacgccggcttcagaatcgccgacgtgcctggagccgccggagtgagcagaggcgcccagagccaccacttccctaccaagctggagctgctgctggccaccttcgagtggctgtacgagcagatcaccgagaggagcagagccagactggccaagctgaagcccgaggacgatgtgatccagcagatgctggatgatgccgccgagttcttcctggacgacgacttcagcatcagcctggacctgatcgtggccgccgacagagaccccgccctgagagagggcatccagaggaccgtggagcggaacagattcgtggtggaggacatgtggctgggagtgctggtgtccagaggcctgagcagagatgacgccgaggacatcctgtggctgatcttcaactctgtgaggggcctggctgtgagaagcctgtggcagaaggacaaggagagattcgagagagtgcggaacagcaccctggagatcgccagagagcgctacgccaagtttaaacggtga

SEQ ID NO: 2 - protein, 203 aa, Pseudomonas putida
MSPKRRTQAERAMETQGKLIAAALGVLREKGYAGFRIADVPGAAGVSRGAQSHHFPTKLELLLATFEWLYEQITERSRARLAKLKPEDDVIQQMLDDAAEFFLDDDFSISLDLIVAADRDPALREGIQRTVERNRFVVEDMWLGVLVSRGLSRDDAEDILWLIFNSVRGLAVRSLWQKDKERFERVRNSTLEIARERYAKFKR

SEQ ID NO: 3 - DNA, 28 bp, Pseudomonas putida
aacaaacagacaatctggtctgtttgta

SEQ ID NO: 4 - DNA, 26 bp, Pseudomonas putida
agaaacaaaccaacctgtctgtatta

SEQ ID NO: 5 - DNA, 1101 bp, Artificial Sequence
  Synthetic Construct
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcaagcttgccgggtcgaggtaggcgtgtacggtgggaggcctatataagcaaccggtataatacaaacagaccagattgtctgtttgttaccggtgtttagtgaaccgggcgcgcctcatatcgcctggagacgccatccacgctgttttgacctccatagaagacaccgggaccgatccagcctccgcggtcactctcttccgcatcgctgtctgcgagggccagctgttgggctcgcggttgaggacaaactcttcgcggtctttccagtactcttggatcggaaacccgtcggcctccgaacggtactccgccaccgagggacctgagccagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagccggcggatggtcgaggtgaggtgtggcaggcttgagatccagctgttggggtgagtactccctctcaaaagcgggcatgacttctgcgctaagattgtcagtttccaaaaacgaggaggatttgatattcacctggccc

SEQ ID NO: 6 - DNA, 25 bp, bacteriophage
tcgagtttacctctggcggtgatag

SEQ ID NO: 7 - DNA, 300 bp, Artificial Sequence
  Synthetic Construct
tcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatag

SEQ ID NO: 8 - DNA, 325 bp, Artificial Sequence
  Synthetic Construct
tcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatag

SEQ ID NO: 9 - DNA, 348 bp, Artificial Sequence
  Synthetic Construct
tcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgactctagataggcgtgtacggtgggaggcctatataagcagagct

SEQ ID NO: 10 - DNA, 373 bp, Artificial Sequence
  Synthetic Construct
tcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgactctagataggcgtgtacggtgggaggcctatataagcagagct

SEQ ID NO: 11 - DNA, 904 bp, Artificial Sequence
  Synthetic Construct
tcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgactctagataggcgtgtacggtgggaggcctatataagcagagctcgtttagtgaaccgtcagatcgcctggagacgccatccacgctgttttgacctccatagaagacaccgggaccgatccagcctccgcggtcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggccc

SEQ ID NO: 12 - DNA, 1372 bp, Artificial Sequence
  Synthetic Construct
tcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgagtttacctctggcggtgatagtcgactctagataggcgtgtacggtgggaggcctatataagcagagctcgtttagtgaaccgtcagatccctggagacgccatccacgctgttttgacctccatagaagacaccgggaccgatcaacctaagcttccaaccggtgtttagtgaaccgggcgcgcctcatatcgcctggagacgccatccacgctgttttgacctccatagaagacaccgggaccgatccagcctccgcggtcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttaacgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctc

SEQ ID NO: 13 - DNA, 1674 bp, Artificial Sequence
  Synthetic Construct
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttgggatgcagccgtcacttagaagtgagtatgagtaccctgttttttctcatgttcaggcagggatgttctcacctgagcttagaacctttaccaaaggtgatgcggagagatgggtagatatctcgaattcttatgactcctccagtatcaaagtcctgaaagggctggatgcggtgcgtaagcgcccgggtatgtatatcggcgacacggatgacggcaccggtctgcaccacatggtattcgaggtggtagataacgctatcgacgaagcgctcgcgggtcactgtaaagaaattatcgtcaccattcacgccgataactctgtctctgtacaggatgacgggcgcggcattccgaccggtattcacccggaagagggcgtatcggcggcggaagtgatcatgaccgttctgcacgcaggcggtaaatttgacgataactcctataaagtgtccggcggtctgcacggcgttggtgtttcggtagtaaacgccctgtcgcaaaaactggagctggttatccagcgcgagggtaaaattcaccgtcagatctacgaacacggtgtaccgcaggccccgctggcggttaccggcgagactgaaaaaaccggcaccatggtgcgtttctggcccagcctcgaaaccttcaccaatgtgaccgagttcgaatatgaaattctggcgaaacgtctgcgtgagttgtcgttcctcaactccggcgtttccattcgtctgcgcgacaagcgcgacggcaaagaagaccacttccactatgaaggcggcccatggatgggccctaaaaagaagcgtaaagtcgccatcgatcagctcaccatggtgtttccttctgggcagatctcaaaccaggccctggccttagcaccgtcctctgccccagtccttgcccagaccatggtcccttcctcagccatggtacctctggctcagcccccagctcctgccccagttctaaccccgggtcctccccagtccctgtctgcacctgttccaaagagcacccaggctggggaaggcacgctgtcggaagccctgctgcacctgcagtttgatgctgatgaagacttgggggccttgcttggcaacagcacagacccaggagtgttcacagacctggcatctgtggacaactcagagtttcagcagctcctgaaccagggtgtgtccatgtctcactccacagctgagcccatgctgatggagtaccctgaagctataactcgcctggtgacagggtcccagaggccccctgacccagctcccacacccctggggacctcggggcttcccaatggtctctccggagatgaagacttctcctccattgcggacatggacttctctgctctgctgagtcagatcagctccagcggccaataa

SEQ ID NO: 14 - protein, 557 aa, Artificial Sequence
  Synthetic Construct
MSTKKKPLTQEQLEDARRLKAIYEKKKNELGLSQESVADKMGMGQSGVGALFNGINALNAYNAALLAKILKVSVEEFSPSIAREIYEMYEAVGMQPSLRSEYEYPVFSHVQAGMFSPELRTFTKGDAERWVDISNSYDSSSIKVLKGLDAVRKRPGMYIGDTDDGTGLHHMVFEVVDNAIDEALAGHCKEIIVTIHADNSVSVQDDGRGIPTGIHPEEGVSAAEVIMTVLHAGGKFDDNSYKVSGGLHGVGVSVVNALSQKLELVIQREGKIHRQIYEHGVPQAPLAVTGETEKTGTMVRFWPSLETFTNVTEFEYEILAKRLRELSFLNSGVSIRLRDKRDGKEDHFHYEGGPWMGPKKKRKVAIDQLTMVFPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQ

SEQ ID NO: 15 - protein, 181 aa, Mus musculus
PSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISS

SEQ ID NO: 16 - DNA, 56 bp, Oryctolagus cuniculus
aataaaggaaatttattttcattgcaatagtgtgttggaattttttgtgtctctca

SEQ ID NO: 17 - DNA, 225 bp, Bos taurus
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 18 - DNA, 122 bp, Simian virus 40
aacttgtttattgcagcttataatggttacaaataaagcaatagcatcacaaatttcacaaataaagcatttttttcactgcattctagttgtggtttgtccaaactcatcaatgtatctta

SEQ ID NO: 19 - DNA, 1536 bp, Artificial Sequence
  Synthetic Construct
atgaaatgtctgctgtacctggcattcctgtttatcggagtcaactgcaagtttactatcgtcttcccccacaatcagaaaggcaattggaagaacgtgccaagcaattaccactattgccccagctcctctgacctgaactggcataatgatctgatcggcaccgccctgcaggtcaagatgcccaaatcccacaaggccatccaggctgacgggtggatgtgccatgcttctaaatgggtgaccacatgtgacttccggtggtacggaccaaagtatatcactcatagcattcgctccttcaccccctccgtggagcagtgcaaagagtctattgaacagaccaagcaggggacatggctgaaccctggatttccccctcagtcctgtgggtacgccacagtcactgacgctgaggcagtgatcgtccaggtgacaccacaccatgtcctggtggacgagtatactggggaatgggtggattcacagttcattaacggaaaatgcagcaattacatctgtcctacagtccacaactctactacctggcatagtgattataaggtgaaaggcctgtgcgatagcaatctgatctccatggacattactttctttagtgaggatggcgaactgagttcactggggaaggagggaaccggctttcggagcaattacttcgcatatgaaacaggcgggaaagcctgcaagatgcagtactgtaaacactggggagtccgcctgccatctggcgtgtggttcgagatggcagacaaggatctgtttgccgctgcacgattcccagagtgccccgaaggcagctccatctctgcccccagtcagacttcagtggacgtgagcctgattcaggatgtggagagaatcctggactacagtctgtgccaggaaacctggtcaaaaattagggctggcctgcctatctcaccagtggacctgagctatctggctcccaaaaaccctgggactggacccgccttcaccatcattaatgggacactgaagtacttcgagacccggtatatcagagtggacattgccgctcctatcctgagccgaatggtgggcatgatctccgggacaactaccgagcgggaactgtgggacgattgggctccttacgaggatgtcgaaattggaccaaacggcgtgctgaggacatctagtggctacaaatttcctctgtatatgatcggccacgggatgctggactctgatctgcatctgtcaagcaaggcacaggtgttcgagcacccccatatccaggacgcagcctctcagctgcctgacgatgaaagtctgttctttggggataccggactgagcaaaaatccaattgagctggtggaaggatggttttcctcttggaagagttcaatcgcctccttctttttcatcattggactgatcattggcctgttcctggtcctgcgggtgggcattcacctgtgcatcaagctgaaacataccaagaaaagacagatttacaccgacattgagatgaacagactgggcaagtga

SEQ ID NO: 20 - protein, 511 aa, Vesicular stomatitis virus
MKCLLYLAFLFIGVNCKFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

(19) United States
(12) Patent Application Publication
(10) Pub. No.: US 2025/0346919 A1
(43) Pub. Date: Nov. 13, 2025

(54) Tightly-Regulated Inducible Expression System for Production of Biologics Using Stable Cell Lines

(71) Applicant: National Research Council of Canada, Ottawa (CA)

(72) Inventors: Rénald GILBERT, Montréal (CA); Sophie BROUSSAU, Varennes (CA); Claire GUILBAULT, Montréal (CA); Mélanie LECLERC, Carignan (CA); Viktoria LYTVYN, Pierrefonds (CA); Mélanie SIMONEAU, Montréal (CA)

(73) Assignee: National Research Council of Canada, Ottawa, ON (CA)

(21) Appl. No.: 18/259,088

(22) PCT Filed: Jan. 6, 2022
(86) PCT No.: PCT/CA2022/050010

§ 371 Date: Jun. 23, 2023

Related U.S. Application Data
(60) Provisional application No. 63/134,816, filed on Jan. 7, 2021.

(51) Int. Cl. C12N 15/86; C12P 19/34; C12P 21/02

(52) U.S. Cl. CPC C12N 15/86; C12P 19/34; C12P 21/02; C12N 2740/16043; C12N 2740/16052; C12N 2750/14143; C12N 2750/14152; C12N 2830/002

(57) ABSTRACT

The disclosure pertains to tightly regulated inducible expression systems useful for the inducible production of one or more RNAs or proteins of interest, including the production of biologics such as recombinant proteins, vaccines, or viral vectors. Also provided are cell lines and kits useful for the production of said RNAs or proteins, as well as methods for making said cell lines and methods for inducing production of said RNAs or proteins.

### CROSS-REFERENCE TO RELATED APPLICATIONS

This application is a national stage entry of PCT application no. PCT/CA2022/050010, filed Jan. 6, 2022, which claims the benefit of priority to U.S. Provisional Application No. 63/134,816, filed Jan. 7, 2021, the contents of each of which is incorporated herein by reference in its entirety.

### REFERENCE TO AN ELECTRONIC SEQUENCE LISTING

The contents of the electronic sequence listing (2018-099-03_SL_8Nov.2023_ST25.txt; Size: 26,976 bytes; and Date of Creation: Nov. 8, 2023) is herein incorporated by reference in its entirety.

### FIELD

The present disclosure relates to gene expression systems for the inducible expression of one or more RNAs or proteins of interest in mammalian cells. Also disclosed are cell lines and methods for inducible production of biologics such as recombinant proteins, vaccines, or viral vectors in mammalian cells.

### BACKGROUND

Various biologic products, such as recombinant proteins, vaccines and viral vectors, are often produced for clinical applications using cultured mammalian cells. The availability of a cell line with the capacity to efficiently produce such biologic products without the need of transfection or infection, greatly facilitates the manufacturing process. However, it is often difficult to generate such cell lines because some of the components making up these biologic products are cytotoxic. Consequently, the constitutive synthesis of these components prevents the cells from growing properly. These cells are unstable or produce low amounts of product. They are therefore not suited for manufacturing.

To produce biologic products that are cytotoxic using a stable cell line, the transcription of their genes needs to be regulated using an inducible expression system, such as the Tetracycline gene-switch (Gossen and Bujard, 1992), the cumate gene-switch (Mullick et al., 2006) or the coumermycin gene-switch (Zhao et al., 2003). With such an inducible expression system, transcription of the gene encoding the biologic product is inactive (the expression system is turned off) during isolation and growth of the cells. When synthesis of the biologic product is needed, transcription is activated (the expression system is turned on) by adding an inducer (doxycycline or cumate, for example). In order to use an inducible expression system, the cells must have integrated into their chromosomes a gene or genes encoding the regulatory elements of the gene-switch, such as a transactivator and/or repressor.

Several inducible expression systems have been developed (for example, the cumate, tetracycline and coumermycin gene-switches). The major drawbacks of some inducible expression systems are their leakiness (the gene of interest is transcribed at low levels without induction), and/or modest efficacy (when induced, the expression system confers only weak gene expression).

### SUMMARY

The present disclosure provides expression systems for the inducible expression of one or more RNAs or proteins of interest by combining the coumermycin gene-switch (Zhao et al., 2003) with the cumate gene-switch (Mullick et al., 2006) to provide dual regulation and reduced leakiness of target gene expression. The present inventors have demonstrated this dual switch expression system, using the gene for CymR regulated by a constitutive promoter, and the gene for λR-GyrB under the control of a cumate-inducible promoter. The transcription of λR-GyrB was therefore controlled by the cumate gene-switch. In the absence of cumate, the CymR repressor bound to a cumate-inducible promoter, CMV5CuO, and prevented transcription of λR-GyrB. Transcription of the λR-GyrB gene (and therefore production of the λR-GyrB transactivator) was induced by the addition of cumate, which released the CymR from the CMV5CuO promoter. In this gene expression system, the transcription of the gene(s) encoding the biologic product(s) to be produced was regulated by the coumermycin-inducible promoter 12xlambda-CMVmin (or 12xlambda-TPL, or variations of these promoters). This promoter was activated by the binding of a dimer of XR-GyrB. The XR-GyrB formed a dimer in the presence of coumermycin. In the absence of coumermycin, XR-GyrB would remain as a monomer, would not bind to 12xlambda-CMVmin and consequently would not activate the transcription from 12xlambda-CMVmin. In summary, in the dual cumate/coumermycin gene-switch, induction was achieved by adding two inducers: cumate (which releases the inhibition by CymR and allows the synthesis of λR-GyrB), and coumermycin, with allowed the dimerization of λR-GyrB and transcription from 12xlambda-CMVmin.

An aspect of the disclosure includes an inducible expression system comprising: a first expression cassette comprising a nucleic acid molecule encoding a cumate repressor protein operably linked to a constitutive promoter and a polyadenylation signal; a second expression cassette comprising a nucleic acid molecule encoding a coumermycin chimeric transactivator protein operably linked to a cumate-inducible promoter and a polyadenylation signal; and a third expression cassette comprising: (i) a nucleic acid molecule comprising a coumermycin-inducible promoter, a cloning site, and a polyadenylation signal, wherein the cloning site is for insertion of a nucleic acid molecule encoding a first RNA or protein of interest in operable linkage with the coumermycin-inducible promoter and the polyadenylation signal, or (ii) a nucleic acid molecule encoding a first RNA or protein of interest operably linked to a coumermycin-inducible promoter and a polyadenylation signal.

In an embodiment, the constitutive promoter is selected from the group consisting of human Ubiquitin C (UBC) promoter, human Elongation Factor 1 alpha (EF1A) promoter, human phosphoglycerate kinase 1 (PGK) promoter, simian virus 40 early promoter (SV40), beta-actin promoter, cytomegalovirus immediate-early promoter (CMV), hybrid CMV enhancer/beta-actin promoter (CAG), and variants thereof.

In an embodiment, the cumate repressor protein comprises the amino acid sequence set forth in SEQ ID NO: 2 or a functional variant thereof, or is encoded by a nucleic acid molecule comprising the nucleotide sequence set forth in SEQ ID NO: 1 or a functional variant thereof.

In an embodiment, the cumate-inducible promoter comprises the nucleotide sequence set forth in SEQ ID NO: 5 or a functional variant thereof.

In an embodiment, the coumermycin chimeric transactivator protein comprises the amino acid sequence set forth in SEQ ID NO: 14 or a functional variant thereof, or is encoded by a nucleic acid molecule comprising the nucleotide sequence set forth in SEQ ID NO: 13 or a functional variant thereof.

In an embodiment, the coumermycin-inducible promoter comprises the nucleotide sequence set forth in SEQ ID NO: 9 or a functional variant thereof, or comprises the nucleotide sequence set forth in SEQ ID NO: 10 or a functional variant thereof.

In an embodiment, the coumermycin-inducible promoter further comprises a tripartite leader (TPL) and/or a major late promoter (MLP) enhancer. In an embodiment, the coumermycin-inducible promoter comprises the nucleotide sequence set forth in SEQ ID NO: 11 or a functional variant thereof.

In an embodiment, the coumermycin-inducible promoter further comprises a human beta-globin intron. In an embodiment, the coumermycin-inducible promoter comprises the nucleotide sequence set forth in SEQ ID NO: 12 or a functional variant thereof.

In an embodiment, the third expression cassette comprises the nucleic acid molecule encoding the first RNA or protein of interest operably linked to the coumermycin-inducible promoter and the polyadenylation signal. In an embodiment, the third expression cassette encodes a recombinant protein.

In an embodiment, the expression system further comprises a fourth expression cassette comprising a nucleic acid molecule encoding a second RNA or protein of interest operably linked to a promoter and a polyadenylation signal.

In an embodiment, the expression system further comprises a fifth expression cassette comprising a nucleic acid molecule encoding a third RNA or protein of interest operably linked to a promoter and a polyadenylation signal.

In an embodiment, the promoter of the fourth and/or fifth expression cassette is a coumermycin-inducible promoter.

In an embodiment, the promoter of the fourth and/or fifth expression cassette is a constitutive promoter.

In an embodiment, the expression system encodes one or more components of a viral vector.

In an embodiment, the third expression cassette encodes lentiviral REV protein, the promoter of the fourth expression cassette is a coumermycin-inducible promoter and the fourth expression cassette encodes a viral envelope protein, and the fifth expression cassette encodes a lentiviral Gag/pol. In an embodiment, the viral envelope protein is VSVg, optionally VSVg-Q96H-I57L.

In an embodiment, the third expression cassette encodes a viral envelope protein, the promoter of the fourth expression cassette is a coumermycin-inducible promoter and the fourth expression cassette encodes a lentiviral Gag/pol, and the fifth expression cassette encodes a lentiviral REV protein. In an embodiment, the viral envelope protein is VSVg, optionally VSVg-Q96H-I57L.

In an embodiment, the third expression cassette encodes Rep 40 or Rep 52, the fourth expression cassette encodes Rep 68 or Rep 78, and the fourth expression cassette is under the control of a coumermycin-inducible promoter.

In an embodiment, the third expression cassette encodes Rep52, the fourth expression cassette encodes Rep68, the fifth expression cassette encodes Rep 78, and the fourth and fifth expression cassettes are under the control of a coumermycin-inducible promoter.

In an embodiment, the third expression cassette encodes an antibody heavy chain or a portion thereof, and the fourth expression cassette encodes an antibody light chain or a portion thereof.

Another aspect of the disclosure includes a method of generating a mammalian cell for the production of an RNA or protein of interest. In an embodiment, the method comprises: introducing into a mammalian cell the expression system described herein and a selectable marker, and applying selective pressure to the cell to select for cells that carry the selectable marker, thereby selecting cells that carry the expression system and generating the mammalian cell for the production of the RNA or protein of interest.

In an embodiment, the method further comprises steps of isolating an individual cell carrying the selectable marker and the expression system; and culturing the individual cell to generate a population of cells carrying the selectable marker and the expression system.

In an embodiment, the method comprises: a) introducing into a mammalian cell a first expression cassette of the expression system described herein and a first selectable marker; b) applying selective pressure to the cell to select for cells that carry the first selectable marker, thereby selecting cells that carry the first expression cassette; c) isolating a first individual cell comprising the first expression cassette; d) culturing the first individual cell to obtain a first population of cells comprising the first expression cassette; e) introducing into a cell of the first population of cells a second expression cassette of the expression system described herein and a second selectable marker; f) applying selective pressure to the cell to select for cells that carry the second selectable marker, thereby selecting cells that carry the second expression cassette; g) isolating a second individual cell comprising the second expression cassette; h) culturing the second individual cell to obtain a second population of cells comprising the second expression cassette; i) introducing into a cell of the second population of cells a third expression cassette of the expression system described herein and a third selectable marker; j) applying selective pressure to the cell to select for cells that carry the third selectable marker, thereby selecting cells that carry the third expression cassette; k) isolating a third individual cell comprising the third expression cassette; l) culturing the third individual cell to obtain a third population of cells comprising the third expression cassette, thereby generating the mammalian cell for the production of the RNA or protein of interest.

In an embodiment, a fourth expression cassette, and optionally a fifth expression cassette of the expression system described herein, is introduced into the cell at step i) or after step 1).

In an embodiment, the method of generating a mammalian cell for the production of an RNA or protein of interest comprises: a) introducing into a mammalian cell a first expression cassette of the expression system described herein, a second expression cassette of the expression system described herein, and a first selectable marker; b) applying selective pressure to the cell to select for cells that carry the first selectable marker, thereby selecting cells that carry the first and second expression cassettes; c) isolating a first individual cell comprising the first expression cassette and the second expression cassette; d) culturing the individual cell to obtain a first population of cells comprising the first expression cassette and the second expression cassette; e) introducing into a cell of the first population of cells a third expression cassette of the expression system described herein and a second selectable marker; f) applying selective pressure to the cell to select for cells that carry the second selectable marker, thereby selecting cells that carry the third expression cassette; g) isolating a second individual cell comprising the third expression cassette; h) culturing the second individual cell to obtain a second population of cells comprising the third expression cassette, thereby generating the mammalian cell for the production of the RNA or protein of interest.

In an embodiment, a fourth expression cassette of the expression system described herein, and optionally a fifth expression cassette of the expression system described herein, is introduced into the cell at step e) or after step h).

In an embodiment, the expression system or one or more expression cassettes of the expression system described herein are introduced into the cell by transfection, transduction, infection, electroporation, sonoporation, nucleofection, or microinjection.

Another aspect includes a cell comprising the expression system described herein, or generated by the methods described herein.

In an embodiment, the cell is a human cell, optionally a Human Embryonic Kidney (HEK)-293 cell or a derivative thereof, a Chinese Hamster Ovary (CHO) cell or a derivative thereof, a VERO cell or a derivative thereof, a HeLa cell or a derivative thereof, an A549 cell or a derivative thereof, a stem cell or a derivative thereof, or a neuron or a derivative thereof.

Another aspect includes methods of producing an RNA or protein of interest. In an embodiment, the method comprises culturing a cell comprising the expression system described herein in the presence of a cumate effector molecule and a coumermycin effector molecule, wherein a third expression cassette of the expression system of the cell encodes the RNA or protein of interest and wherein the RNA or protein of interest is produced.

In an embodiment, the cumate effector molecule is cumate, optionally the cumate is present at a concentration of about 1 to about 200 μg/ml, about 50 to about 150 μg/ml, or about 100 μg/ml.

In an embodiment, the coumermycin effector molecule is coumermycin, optionally the coumermycin is present at a concentration of about 1 to about 30 nM, about 5 to about 20 nM, or about 10 nm.

In an embodiment, the cell is grown in suspension and/or in the absence of serum.

An aspect includes a viral packaging cell comprising the expression system described herein. In an embodiment, the viral packaging cell is a lentiviral packaging cell. In another embodiment, the viral packaging cell is an adeno-associated virus (AAV) packaging cell.

In an embodiment, the viral packaging cell further comprises a viral construct carrying a gene of interest.

Another aspect includes a method of producing a viral vector, the method comprising: introducing into the viral packaging cell described herein a viral construct carrying a gene of interest; and culturing the cell in the presence of a cumate effector molecule and a coumermycin effector molecule, thereby producing the viral vector.

In an embodiment, the cumate effector molecule is cumate and/or the coumermycin effector molecule is coumermycin.

In an embodiment, the viral packaging cell is grown in suspension and/or in the absence of serum.

In an embodiment, a selectable marker is introduced into the viral packaging cell with the viral construct, and the method further comprises applying selective pressure to select for cells that carry the selectable marker, thereby selecting cells that carry the viral construct, and optionally isolating an individual cell comprising the viral construct and culturing the individual cell comprising the viral construct to obtain a population of cells comprising the viral construct.

In an embodiment, the viral packaging cell is a lentiviral packaging cell and the viral construct is a lentiviral construct.

In an embodiment, the viral packaging cell is an AAV packaging cell and the viral construct is an AAV construct.

A further aspect includes a method of generating an expression-ready mammalian cell line. In an embodiment, the method comprises: introducing into a mammalian cell a first expression cassette of the expression system described herein, a second expression cassette of the expression system described herein, and a first selectable marker; applying selective pressure to the cell to select for cells that carry the first selectable marker, thereby selecting cells that carry the first and second expression cassettes; isolating an individual cell comprising the first and second expression cassettes; and culturing the individual cell to generate a cell line comprising the first and second expression cassettes, thereby generating the expression-ready mammalian cell line.

In an embodiment, the method of generating an expression-ready mammalian cell line comprises: introducing into a mammalian cell a first expression cassette of the expression system described herein and a first selectable marker; applying selective pressure to the cell to select for cells that carry the first selectable marker, thereby selecting cells that carry the first expression cassette; isolating a first individual cell comprising the first expression cassette; culturing the first individual cell to obtain a first population of cells comprising the first expression cassette; introducing into a cell of the first population of cells a second expression cassette of the expression system described herein and a second selectable marker; applying selective pressure to the cell to select for cells that carry the second selectable marker; isolating a second individual cell comprising the second expression cassette; and culturing the second individual cell to obtain a second population of cells comprising the second expression cassette, thereby generating the expression-ready mammalian cell line.

Another aspect includes a mammalian cell comprising a first expression cassette of the expression system described herein and a second expression cassette of the expression system described herein.

In an embodiment, the cell is a human cell, optionally a Human Embryonic Kidney (HEK)-293 cell or a derivative thereof.

A further aspect includes a method of producing an RNA or protein of interest, the method comprising: introducing into a cell comprising a first expression cassette of the expression system described herein, a second expression cassette of the expression system described herein, a third expression cassette of the expression system described herein and a selectable marker; applying selective pressure to the cell to select for cells that carry the selectable marker, thereby selecting cells that carry the first, second, and third expression cassettes of the expression system; optionally isolating an individual cell comprising the first, second, and third expression cassettes, and culturing the individual cell to generate a population of cells comprising the first, second, and third expression cassettes; and culturing the cell comprising the first, second, and third expression cassettes in the presence of a cumate effector molecule and a coumermycin effector molecule, wherein the RNA or protein of interest is produced. In an embodiment, the cumate effector molecule is cumate and/or the coumermycin effector molecule is coumermycin. In an embodiment, the cell is grown in suspension and/or in the absence of serum.

Yet another aspect of the disclosure includes a kit comprising the expression system described herein. In an embodiment, the kit comprises: a first plasmid comprising a first expression cassette of the expression system described herein; a second plasmid comprising a second expression cassette of the expression system described herein; and a third plasmid comprising a third expression cassette of an expression system described herein. In an embodiment, the kit comprises a cell comprising a first expression cassette and a second expression cassette of the expression system described herein, and a plasmid comprising a third expression cassette of an expression system described herein.

In an embodiment, the third expression cassette comprises a coumermycin-inducible promoter, a cloning site, and a polyadenylation signal, wherein the cloning site is for insertion of a nucleic acid molecule encoding a first RNA or protein of interest in operable linkage with the coumermycin-inducible promoter and the polyadenylation signal. In an embodiment, the third expression cassette comprises a nucleic acid molecule encoding a first RNA or protein of interest operably linked to a coumermycin-inducible promoter and a polyadenylation signal.

In an embodiment, the cell comprising a first expression cassette and a second expression cassette of the expression system described herein, further comprises a third, fourth, and/or fifth expression cassette of the expression system described herein, wherein the third, fourth, and/or fifth expression cassettes encode an RNA or protein of interest.

In an embodiment, the kit comprises a viral packaging cell comprising the expression system described herein, and a viral construct.

In an embodiment, the kit further comprises a cumate effector molecule, optionally cumate, and/or a coumermycin effector molecule, optionally coumermycin.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### BRIEF DESCRIPTION OF THE DRAWINGS

Further objects, features and advantages of the disclosure will become apparent from the following detailed description taken in conjunction with the accompanying figures showing illustrative embodiments of the disclosure, in which:

FIG. 1 shows a schematic of the mechanism of gene regulation of an embodiment of the cumate/coumermycin gene-switch. A) 293SF-CymR/λR-GyrB cells were engineered to constitutively produce the repressor of the cumate gene-switch (CymR). The cells also contain the gene for the coumermycin chimeric transactivator (λR-GyrB) under the control of the CMV5CuO promoter. In the absence of cumate, CymR binds to the CMV5CuO promoter and prevents transcription of λR-GyrB. Addition of cumate releases CymR from the promoter and λR-GyrB can be transcribed. B) In the presence of coumermycin, λR-GyrB forms a dimer that binds to the 12xlambda operator (12xλOp) and activates transcription of the transgene of interest. Novobiocin can be added to the cells to dissociate the λR-GyrB dimers and thereby prevent transcription.

FIG. 2 shows a diagram of constructs used to make 293SF-CymR, 293SF-CymR/λR-GyrB and 293SF-CymR/rcTA cells. A) CymR construct used to make 293SF-CymR, 293SF-CymR/λR-GyrB and 293SF-CymR/rcTA cells. The coding sequence of CymR repressor is controlled by a strong constitutive promoter (CMV5). B) λR-GyrB construct used to make the 293SF-CymR/λR-GyrB cells. The coding sequence of λR-GyrB is controlled by the CMV5CuO promoter. C) rcTA construct used to make the 293SF-CymR/rcTA cells. The coding sequence of rcTA is controlled by the CMV5CuO promoter. pA: polyadenylation signal.

FIG. 3 shows a diagram of the transfer vectors encoding the LV used in this study. A) Transfer vector for LV-CMV5CuO-GFP. B) Transfer vector for LV-12xlambda-TPL-GFP. C) Transfer vector for LV-CR5-GFP. D) Transfer vector for LV-CMV-GFP. Note: the transfer vectors shown in A), B) and C) produce conditional Self-Inactivating lentivirus (cSIN). 5′LTR and 3′LTR: Long Terminal Repeat located at the 5′ or 3′ end of the lentivirus respectively; CMV: CMV promoter; R: R region of the LTR; U5: U5 region of the LTR: Tet: tetracycline promoter; ψ: encapsidation signal; RRE: Rev Responsive Element; cPPT: Central Polypurine Track; GFP: Green Fluorescent Protein; WPRE: Woodchuck Hepatitis Virus Posttranscriptional Regulatory Element; SD and SA: splice donor and acceptor respectively; CMV5CuO and CR5 cumate regulated promoter; 12xλ-TPL: 12xLambda-TPL Coumermycin regulated promoter

FIG. 4 shows a schematic of the mechanism of gene regulation in 293SF-CymR/rcTA cells. A) The 293SF-CymR/rcTA cells were engineered to constitutively produce the repressor of the cumate gene-switch (CymR). The cells also contain the gene for the reverse transactivator (rcTA) under the control of the CMV5CuO promoter. In the absence of cumate, CymR binds to the CMV5CuO promoter and prevents transcription of rcTA gene. Addition of cumate releases CymR from the promoter and allows transcription of rcTA. B) In the presence of cumate, rcTA binds to the CR5 promoter and activates transcription of the transgene of interest (reporter).

FIG. 5 shows that the dual coumermycin/cumate gene-switch provides a better induction level compared to the cumate gene-switch. A) Clones of 293SF-CymR (198-2, 198-10, 169-4, 169-C1, CA7), B) Clones of 293SF-CymR/rcTA (G3 and G11) and C) Clones of 293SF-CymR/λR-GyrB (7-2, 7-3, 7-10) were transduced with LV-CMV5CuO-GFP, LV-CR5-GFP and LV-12xlambda-TPL-GFP respectively. After transduction, the level of GFP expression was analyzed by flow cytometry. The fluorescent indexes of the cell population in the absence (Off) and in presence of inducers (On) were compared. The On/Off ratio for each clone is indicated by a number above the bars (B,C) or as a line (A). The On/Off ratio for the 293SF-CymR, 293SF-CymR/rcTA and 29SF-CymR/λR-GyrB clones varied between 15-30, 60-100 and 2621-3877 respectively. 293SF are 293SF cells (without the switch) transduced with the LV; T0 and T2 are cells maintained for 1 and 8 weeks in culture respectively.

FIG. 6 shows diagrams of the expression cassettes used to construct the packaging cell line for LV. To construct LV packaging cell lines, the following components were integrated into the chromosomes of 293SF-CymR/λR-GyrB: i) the HIV Rev gene under the regulation of the coumermycin inducible promoter, 13xlambda-TPL; ii) the HIV Gag/pol gene regulated by the constitutive hybrid CMV enhancer/β-actin promoter (CAG) or by 11xlambda-hbgmin promoter; and iii) the Vesicular Stomatitis Virus glycoprotein gene (VSVg) regulated by 13xlambda-TPL. Addition of cumate and coumermycin induces the transcription of Rev, VSVg and Gag/pol (when regulated by 11xlambda-hgbmin). Note: the presence of Rev is needed for the nuclear export of unprocessed Gag/pol RNA. Hence, the efficient synthesis of Gag/pol polypeptide depends on the expression of Rev.

FIG. 7 shows production of lentivirus after transfection of packaging cells. Clones of packaging cells grown in suspension culture using serum free medium were transfected with a transfer vector (a plasmid) for LV-CMV-GFP (FIG. 3D). The cells were induced with cumate and coumermycin and the culture medium was harvested at three days post-transfection. The LV in the culture medium was titrated by flow cytometry after transduction of HEK293 cells. The bars are the infectious titers (Transducing units [TU] per ml) in the culture medium. The transfection efficiency (% of GFP positive cells) is indicated in A) using light gray squares. A) Clones of packaging cells generated using a plasmid encoding 11xlambda-hbgmin-Gag/pol. B) Clones of packaging cells generated using a plasmid encoding CAG-Gag/pol. Note: Both packaging cell lines (A and B) gave rise to clones capable of producing LV at a titer equal or greater than 1.0×10⁷ TU/ml.

FIG. 8 shows lentivirus production by producer clones derived from the packaging cell line. LV producer clones were generated by transfecting the packaging cells (clone 3D4, FIG. 7B) with a transfer vector (a plasmid) encoding LV-CMV-GFP (FIG. 3D). The resulting LV (LV-CMV-GFP) expresses GFP under the control of the constitutive CMV promoter. Clones with stably integrated plasmid were isolated and grown in suspension culture using serum-free medium. LV-CMV-GFP production was induced following the addition of cumate and coumermycin to the culture medium. The titer of LV-CMV-GFP in the culture medium was measured at three days post-induction after transduction of HEK293 cells and quantification of GFP positive cells by flow cytometry. The titer is expressed as transducing units (TU) per ml of non-concentrated culture medium. Note: several clones (1E9, 3E9, 2G11, 1F3, 2C8 and 1E8) produce LV-CMV-GFP at a titer greater than 1.0×10⁸ TU/ml of culture medium.

FIG. 9 shows regulation of LV gene expression in the packaging cells. Western blot analysis for the expression of VSVg (A), Rev (B) and Gag (C) by the packaging cells (clone 3D4, FIG. 7), following induction with cumate and coumermycin. The same amount of cell lysate was used for each sample. Cells were harvested before induction (0) and at 24, 48 and 72 hrs after induction in the absence or presence of sodium butyrate (added 18 hrs post-induction). 293SF-CymR/λR-GyrB cells were used as a negative control (CT). The positions of VSVg, Rev, the Gag polyprotein (GAG PP) and p24 are indicated with arrows. MW: molecular weight marker in kDa. Note the presence of a non-specific bands (*) in the negative control when using the anti-VSVg antibody.

FIG. 10 shows a schematic of the components used to produce AAV. A) Constructs expressing Rep52, Rep68, and Rep78 that were used to generate the 293SF-Rep cells. Each of the Rep genes is controlled by the 13xlambda-TPL promoter (13xλ-TPL). B) Plasmids used to produce AAV by transient transfection of 293SF-Rep. pCMV-CAP encodes the CAP gene of AAV regulated by the CMV promoter. pAAV-GFP is the expression vector that produces GFP regulated by CMV. pHelper carries the helper gene of adenovirus.

FIG. 11 shows a western blot of REP protein expression by 293SF-Rep clones. Cell lysate of stably transfected 293SF-Rep clones (clones 13, 18, 35 and 36) was analysed by western blot using an antibody against REP. Expression was induced using different concentrations of cumate and coumermycin. Uninduced cells served as negative control. The position of Rep78, 68 and 52 is indicated by arrows. Note: no significant expression of REP is observed without induction. MW: molecular weight marker.

FIG. 12 shows optimization of AAV production after transient transfection of 293SF-Rep cells (clone 13). 293SF-Rep cells grown in suspension culture and in serum-free medium were transfected with different quantities (expressed as g/ml) of pCMV-CAP (pVR46-CAP), pHelper and pAAV-GFP (see FIG. 10). Following transfection, the cells were induced with cumate and coumermycin for three days. Cell lysate containing the AAV produced was used to transduce HEK293 cells, which were subsequently analysed by flow cytometry. The data is expressed as infectious virus particles (IVP) per ml of cell culture. The best conditions were #5 and #16, which resulted in a titer of 2.5×10⁷ IVP/ml.

FIG. 13 shows the sequence of the promoter 12xLambda-CMVmin (SEQ ID NO: 9) (top strand) and complementary sequence (bottom strand). The position of the 12 copies of the lambda operator (12xlambdaOP) and of the CMV minimal promoter is indicated under the nucleotide sequence of 12xLambda-CMVmin.

FIG. 14 shows the sequence of the promoter 13xLambda-CMVmin (SEQ ID NO: 10) (top strand) and complementary sequence (bottom strand). The position of the 13 copies of the lambda operator (13xlambdaOP) and of the CMV minimal promoter is indicated under the nucleotide sequence of 13xLambda-CMVmin.

FIG. 15 shows the sequence of the promoter 13xLambda-TPL (SEQ ID NO: 11) (top strand) and complementary sequence (bottom strand). The position of the 13 copies of the lambda operator (13xlambdaOP), the CMV minimal promoter, the adenovirus tripartite leader (TPL) and the enhancer of the adenovirus major late promoter (MLP Enhancer) within a small intron are indicated.

FIG. 16. Shows the sequence of the promoter 11xlambda-hbgmin (SEQ ID NO: 12) (top strand) and complementary sequence (bottom strand). The position of the 11 copies of the lambda operator (11xlambdaOP), the CMV minimal promoter, the adenovirus tripartite leader (TPL), enhancer of the adenovirus major late promoter (MLP Enhancer), the human 8-globin intron (hBglobin-delta intron) and splice site donor and acceptor of the chimeric intron are indicated.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. DEFINITIONS

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated.

The term “about” as used herein may be used to take into account experimental error and variations that would be expected by a person having ordinary skill in the art. For example, “about” may mean plus or minus 10%, or plus or minus 5%, of the indicated value to which reference is being made.

As used herein the singular forms “a”, “an”, and “the” include plural references unless the context clearly dictates otherwise.

The phrase “and/or”, as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

### II. EXPRESSION SYSTEMS

The use of a dual coumermycin/cumate gene-switch was found to provide tightly regulated expression (higher On/Off ratio) of a protein of interest compared to use of a cumate switch alone. Accordingly, provided herein is an expression system comprising a dual coumermycin/cumate gene-switch useful for tightly regulated inducible expression of an RNA or protein of interest. Accordingly, herein provided is an expression system comprising a) a first expression cassette comprising a nucleic acid molecule encoding a cumate repressor protein operably linked to a constitutive promoter and a polyadenylation signal; b) a second expression cassette comprising a nucleic acid molecule encoding a coumermycin chimeric transactivator protein operably linked to a cumate-inducible promoter and a polyadenylation signal; and c) a third expression cassette comprising (i) a coumermycin-inducible promoter, a cloning site, and a polyadenylation signal, wherein the cloning site is for insertion of a nucleic acid molecule encoding a first RNA or protein of interest in operable linkage with the coumermycin-inducible promoter and the polyadenylation signal or (ii) a nucleic acid molecule encoding at least one RNA or protein of interest operably linked to a courmermycin-inducible promoter and a polyadenylation signal.

The term “expression cassette” refers to a DNA molecule encoding an RNA or protein operably linked to a promoter and a polyadenylation signal, such that certain portions of the expression cassette are capable of being transcribed into RNA (such as antisense RNA, Long non-coding RNA or for the genome of a virus such as a lentivirus) and/or as a messenger RNA that is subsequently translated into protein by cellular machinery. The term “expression cassette” is also used to refer to a nucleic acid molecule comprising a promoter, a polyadenylation signal, and a cloning site for insertion of a nucleic acid molecule encoding an RNA or protein of interest in operable linkage with the promoter and the polyadenylation signal.

The term “nucleic acid molecule” and its derivatives, as used herein, are intended to include unmodified DNA or RNA or modified DNA or RNA. For example, the nucleic acid molecules or polynucleotides of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “cloning site” as used herein refers to a portion of a nucleic acid molecule into which a nucleic acid molecule of interest may be inserted, or to which a nucleic acid molecule of interest may be joined, using recombinant DNA technology (cloning). In the context of an expression cassette, the cloning site may be located between the promoter and the polyadenylation signal, such that a nucleic acid molecule of interest may be cloned into the expression cassette in operable linkage with the promoter and the polyadenylation site. Several cloning techniques are known to the skilled person and the cloning site will include the necessary characteristics (such as restriction endonuclease site(s), recombinase recognition site(s), or blunt or overhanging end(s)) to allow insertion of the nucleic acid molecule of interest at the cloning site. The cloning site may, for example, be a multiple cloning site (MCS) or polylinker region comprising a plurality of unique restriction enzyme recognition sites to allow a nucleic acid molecule of interest to be inserted. Alternately, or in addition, the cloning site may include one or more recombinase recognition sites to allow DNA insertion by recombinational cloning; employing site-specific recombinase(s), such as Integrase or Cre Recombinase, to catalyze DNA insertion. Examples of recombinational cloning systems include Gateway® (Integrase), Creator™ (Cre Recombinase), and Echo Cloning™ (Cre Recombinase). For some cloning strategies, an expression cassette or vector may be provided as a linear molecule, allowing blunt or overhanging ends of a nucleic acid molecule of interest to be joined to blunt or overhanging ends of the expression cassette or vector, for example by ligation or polymerase activity, thus forming a circular molecule. In this case, the blunt or overhanging ends of the expression cassette or vector may together be viewed as the cloning site. Such an approach is commonly used to clone PCR products.

The term “operably linked” as used herein refers to a relationship between two components that allows them to function in an intended manner. For example, where a DNA encoding an RNA of interest is operably linked to a promoter, the promoter actuates expression of the RNA encoded therein.

The term “promoter” or “promoter sequence” generally refers to a regulatory DNA sequence capable of being bound by an RNA polymerase to initiate transcription of a downstream (i.e. 3′) sequence to generate an RNA. Suitable promoters may be derived from any organism and may be bound or recognized by any RNA polymerase. Suitable promoters will be known to the skilled person. In some expression cassettes, the promoter is a constitutive promoter. Examples of constitutive promoters include human Ubiquitin C (UBC), human Elongation Factor 1alpha (EF1A), human phosphoglycerate kinase 1 (PGK), Vasoactive Intestinal Peptide (VIP), thymidine kinase (tk), Heat Shock Protein (HSP), major late promoter of adenovirus (MLP), mouse mammary tumor virus (MMTV), simian virus 40 early promoter (SV40), beta-actin, cytomegalovirus immediate-early promoter (CMV), hybrid CMV enhancer/beta-actin promoter (CAG), or functional variants thereof. In some expression cassettes, the promoter is an inducible promoter and/or comprises a binding sequence for a transactivator or a repressor that will activate or inhibit transcription respectively. Examples of inducible promoters include cumate-inducible promoters and coumermycin-inducible promoters.

The term “cumate-inducible promoter” as used herein refers to a promoter that is capable of being bound by a cumate repressor protein, such as CymR (SEQ ID NOs: 1 and 2) or a functional variant thereof, in the absence of a cumate effector molecule. Binding of a cumate effector molecule relieves transcriptional repression by the cumate repressor protein, such as CymR. Cumate-inducible promoters are described for example in U.S. Pat. No. 7,745,592 and comprise a minimal promoter sequence (for example a TATA box and adjacent sequence), from for example a mammalian promoter selected from CMV, VIP, tk, HSP, MLP, and MMTV promoters, and at least one CymR operator sequence (CuO). CuO sequences include for example CuO P1 (SEQ ID NO: 4) or CuO P2 as described in U.S. Pat. No. 7,745,592. In some embodiments a CuO having palindromic features such as CuO P2 (SEQ ID NO: 3) is used. In some embodiments of the present disclosure, the CMV5-CuO (SEQ ID NO: 5) or a functional variant thereof may be used.

As used herein, the term “cumate effector molecule” refers to a molecule that relieves transcriptional repression by a cumate repressor protein. Cumate effector molecules include cumate, p-ethylbenzoic acid, p-propylbenzoic acid, cumic acid, p-isobutylbenzoic acid, p-tert-butylbenzoic acid, p-N-dimethylaminobenzoic acid, and p-N-ethylaminobenzoic acid. In an embodiment, the cumate effector molecule is cumate.

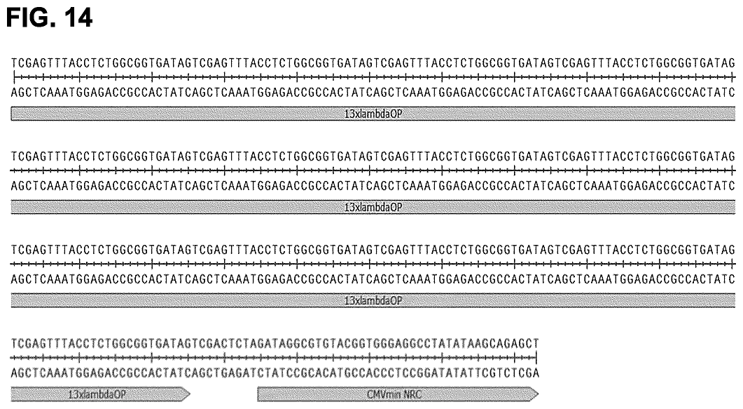

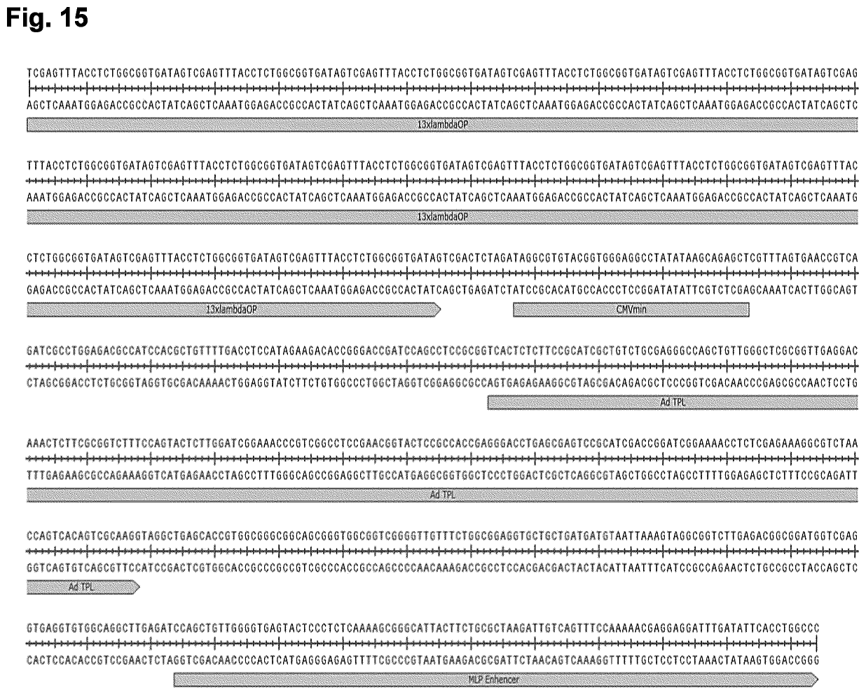

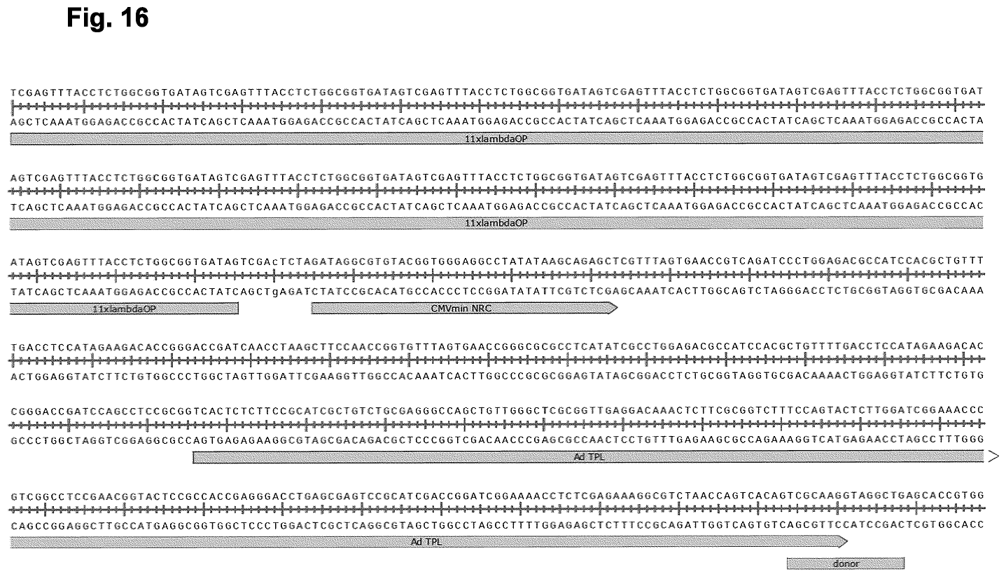

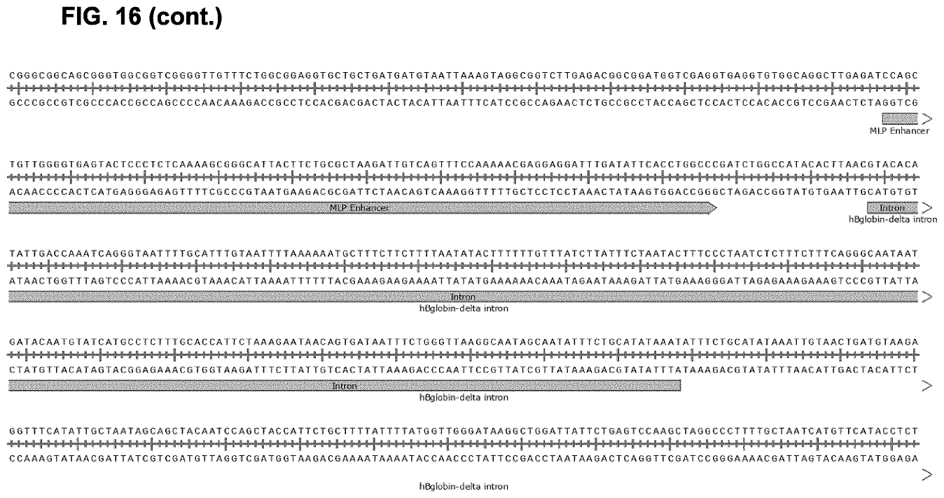

The term “coumermycin-inducible promoter” as used herein refers to a promoter that is capable of being bound by a coumermycin chimeric transactivator protein. Coumermycin-inducible promoters are described, for example, in U.S. Pat. No. 8,377,900 and comprise a minimal promoter sequence (for example a TATA box and adjacent sequence) for example from a constitutive mammalian promoter selected from CMV, VIP, SV40, tk, HSP, PGK, MLP, EF1a, and MMTV promoters, and functional variants thereof, and at least one lambda operator (lambdaOp) sequence (SEQ ID NO: 6). The coumermycin-inducible promoter may comprise for example 1-13 copies of lambdaOp, optionally 11, 12 (SEQ ID NO: 7) or 13 (SEQ ID NO: 8) copies of lambdaOp, or more. In an embodiment the coumermycin-inducible promoter may comprise a minimal CMV promoter and several copies of lambdaOp, such as 11, 12 or 13 copies, for example 12xlambda-CMVmin or 13xlambda-CMVmin as set out in SEQ ID NOs: 9 and 10, respectively, and in FIGS. 13 and 14, or functional variants thereof. In another embodiment, the coumermycin-inducible promoter may comprise several copies of lambdaOp with a minimal CMV promoter and with the tripartite leader (TPL) and major late promoter (MLP) enhancer of adenovirus, for example as for the promoter 13xlambda-TPL as set out in SEQ ID NO: 11 and in FIG. 15, or a functional variant thereof. In another embodiment, the coumermycin-inducible promoter may comprise several copies of lambdaOp associated with other downstream sequences, for example as for the promoter 11xlambda-hbgmin which contains 11 copies of lambdaOp, the minimal CMV promoter, the adenovirus TPL and MLP enhancer and a portion of the human beta-globin intron as set out in SEQ ID NO: 12 and in FIG. 16, or a functional variant thereof.

The term “coumermycin chimeric transactivator protein” as used herein refers to a protein that is capable of binding a coumermycin-inducible promoter in the presence of a coumermycin effector molecule. Binding of a coumermycin effector molecule results in dimerization of the chimeric transactivator protein, such as λR-GyrB, and transcriptional activation from the coumermycin-inducible promoter. Coumermycin chimeric transactivator proteins are described for example in U.S. Pat. No. 8,377,900, and may be constructed by fusing the N-terminal domain of λ repressor (λR) to DNA gyrase B subunit (GyrB) followed by a transcription activation domain, also referred to as a transactivation domain. The N-terminal domain of λR binds as a dimer to lambdaOp. The GyrB domain forms a dimer after binding to coumermycin and therefore promotes the dimerization of N-terminal domain of XR which can then bind to lambdaOp and activate transcription through the activation domain. For simplicity, the coumermycin chimeric transactivator protein may be referred to herein as simply “λR-GyrB”. Suitable transcription activation domains include those from transcription factors NFκB p65, VP16, B42 and Ga14. In an embodiment, the transactivation domain is derived from NFkB p65 as set out in SEQ ID NO: 15. In one embodiment, the coumermycin chimeric transactivator protein has a sequence as set out in SEQ ID NO: 13 or 14, or a functional variant thereof. Suitable coumermycin effector molecules include coumermycin. To provide additional repression of protein expression or prevent leaky expression, the dimerization and activation of coumermycin chimeric transactivator proteins may be inhibited by the addition of an inhibitor such as novobiocin.

The term “polyadenylation signal” or “pA” as used herein refers generally to a polyadenylation signal (pA), which is a site where the transcribed RNA is cleaved and a polyadenylation tail is added, having the effect of terminating transcription of an RNA such as messenger RNA (mRNA). Suitable pAs may be derived from any organism and are known to the skilled person. Examples of pA signals include rabbit beta-globin pA (SEQ ID NO: 16), strong bovine growth hormone pA (BGHpA; SEQ ID NO: 17) and SV40 poly A signal (SEQ ID NO: 18).

The term “functional variant” as used herein includes modifications or chemical equivalents of the nucleic acid sequences or proteins disclosed herein that perform substantially the same function as the nucleic acid molecules or polypeptides disclosed herein in substantially the same way. For example, functional variants of polypeptides disclosed herein include, without limitation, conservative amino acid substitutions.

A “conservative amino acid substitution” as used herein, is one in which one amino acid residue is replaced with another amino acid residue with similar biochemical properties (e.g. charge, hydrophobicity and size). Variants of polypeptides also include additions and deletions to the polypeptide sequences disclosed herein. In addition, variant nucleotide sequences include analogs and derivatives thereof.

In one embodiment, the present disclosure includes functional variants to the nucleic acid sequences disclosed herein. The functional variants include nucleotide sequences that hybridize to the nucleic acid sequences set out above, under at least moderately stringent hybridization conditions.

By “at least moderately stringent hybridization conditions” it is meant that conditions are selected which promote selective hybridization between two complementary nucleic acid molecules in solution. The term “at least moderately stringent hybridization conditions encompasses stringent hybridization conditions and moderately stringent hybridization conditions. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log10 [Na+])+0.41(%(G+C) 600/1), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In some embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm −5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

In another embodiment, the functional variant nucleic acid sequences comprise degenerate codon substitutions or codon-optimized nucleic acid sequences. The term “degenerate codon substitution” as used herein refers to variant nucleic acid sequences in which the second and/or third base of a codon is substituted with a different base that does not result in a change in the amino acid sequence encoded therein. The term “codon-optimized” as used herein refers to a variant nucleic acid molecule comprising one or more degenerate codon substitutions that reflect the codon usage bias of a particular organism.

In another embodiment, the functional variant nucleic acid or protein sequences comprise sequences having at least 50%, or at least 60%, or at least 70%, or at least 80%, or at least 90%, or at least 95% sequence identity to the sequences disclosed herein.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The expression system described herein may be used to express any RNA or protein of interest. Accordingly, in an embodiment the expression system encodes an RNA or protein of interest. The RNA or protein of interest may be cytotoxic or result in reduced cellular growth and/or viability. In one embodiment, the protein of interest is a recombinant protein.

The expression system described herein may be used to express two or more RNAs or proteins of interest, for example for the expression of a complex biologic such as an antibody or viral vector. Accordingly, in one embodiment the expression system comprises one or more additional expression cassettes to allow for the expression of additional RNAs or proteins of interest. The additional RNAs or proteins of interest may be under control of the same regulatory element or a different regulatory element. The additional expression cassette(s) may comprise the same or a different promoter and/or the same or a different pA signal.

In some embodiments, the expression system of the disclosure encodes one or more nucleic acids or proteins involved in the production of a viral vector. The term “viral vector” as used herein is intended to include viral particles or virus-like particles capable of transduction of a target cell. Common viral vectors include, but are not limited to, HIV-derived lentiviral vectors, retroviral vectors, adenoviral vectors, and recombinant adeno-associated virus (AAV) vectors. Other viral vectors may be derived from rhabdovirus (such as vesicular stomatitis virus (VSV)), or herpes virus (such CMV and HSV-1). Accordingly, in an embodiment, the expression system encodes components of a viral vector. Typical components are the structural components of the vectors such as the proteins making the capsid and the envelope of the vector. Other components are the enzymes involved in the replication of the vector RNA or DNA. Such enzymes can be also involved in the synthesis, maturation or transport of the virus RNA. These enzymes can also be involved in the processing and maturation of viral components, as well as in the integration of the genome of the virus into the cell chromosomes. Enzymes that are components of the viral vectors can also be involved in the reverse transcription of the virus genomic RNA into DNA. Other components of the vector can be protein or peptide that regulate the replication, transcription, transport or translation of the genes or gene products of the viral vector. Such factors can also activate or decrease the expression of cellular genes and they can modulate the defense mechanism of the cells against viruses. Some components of the viral vectors, such as the protease of adenovirus and lentivirus (encoded by the gag/pol gene of lentivirus), the Rep proteins of AAV and the envelope glycoprotein of VSV (VSVg) are well known to be toxic to the cells. This list is not exhaustive and other components of the viral vectors or viruses could be toxic if produced constitutively or in too high concentration.

In one embodiment, the viral vector is a lentiviral vector. The expression of REV, Gag/Pol and an envelope protein such as VSVg is involved in the production of lentiviral vectors. Accordingly, in some embodiments, the expression system comprises additional expression cassettes encoding each of REV, Gag/Pol, and an envelope protein such as VSVg, operably linked to a promoter, wherein at least one viral component is under the control of a coumermycin-inducible promoter. Optionally all of the viral components are under the control of a coumermycin-inducible promoter. In some embodiments, Gag/Pol is under the control of a constitutive promoter. In some embodiments, Gag/Pol is under the control of a coumermycin-inducible promoter.

In one embodiment, the viral vector is a recombinant adeno-associated virus (AAV). The expression of Rep proteins is involved in the production of AAV. Accordingly, in some embodiments, the inducible expression system comprises an expression cassette comprising a nucleic acid molecule encoding Rep 40, Rep 52, Rep 68, or Rep 78 operably linked to a coumermycin-inducible promoter. In some embodiments, the expression system comprises one or more additional expression cassettes encoding Rep 40, Rep 52, Rep 68, or Rep 78, operably linked to a promoter, wherein at least one is under the control of a coumermycin-inducible promoter. In some embodiments, the expression system comprises an expression cassette encoding at least one of Rep 40 or Rep 52, and an expression cassette encoding at least one of Rep 68 or Rep 78, wherein at least one is under the control of a coumermycin-inducible promoter. For example, the expression system may comprise Rep 40 and Rep 68, Rep 40 and Rep 78, Rep52 and Rep 68, or Rep 52 and Rep 78. Optionally all of the viral components are under the control of a coumermycin-inducible promoter.

In some embodiments, the gene expression system encodes an antibody fragment, an antibody heavy chain and/or an antibody light chain. The antibody fragment, antibody heavy chain and/or antibody light chain may be encoded in separate expression cassettes, at least one of which is under the control of a coumermycin-inducible promoter.

The term “antibody” as used herein is intended to include monoclonal antibodies, polyclonal antibodies, chimeric and humanized antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Fab, Fab′ and F(ab′)2, scFv, dsFv, ds-scFv, dimers, minibodies, diabodies, bispecific antibody fragments and other fragments can expressed as recombinant proteins.

The basic antibody structural unit is known to comprise a tetramer composed of two identical pairs of polypeptide chains, each pair having one light (“L”) (about 25 kDa) and one heavy (“H”) chain (about 50-70 kDa). The amino-terminal portion of the light chain forms a light chain variable domain (VL) and the amino-terminal portion of the heavy chain forms a heavy chain variable domain (VH). Together, the VH and VL domains form the antibody variable region (Fv) which is primarily responsible for antigen recognition/binding. The carboxy-terminal portions of the heavy and light chains together form a constant region primarily responsible for effector function.

### III. METHODS

The expression system described herein encoding an RNA or protein of interest may be introduced into a mammalian cell for the inducible production of the one or more RNAs or proteins of interest encoded therein. Accordingly, one aspect of the present disclosure is a method of generating a mammalian cell for the inducible production of one or more RNAs or proteins of interest, the method comprising introducing into a mammalian cell the expression system described herein encoding an RNA or protein of interest, introducing into the cell a selectable marker, and applying selective pressure to the cell to select for cells that carry the selectable marker, thereby selecting cells that carry the expression system.

Various mammalian cells may be used for production of the one or more RNAs or proteins of interest. Suitable cells are well known in the art and may include without limitation Chinese Hamster Ovary (CHO) cells, human embryonic kidney 293 (HEK293) cells, VERO cells, HeLa cells, A549 cells, stem cells, and neurons. In some embodiments the cell is a HEK293 cell, optionally a 293SF-3F6 cell as described in U.S. Pat. No. 6,210,922. In some embodiments, the cells are grown in suspension and/or may be grown in the absence of serum.

The expression system may be introduced into the cell by any suitable method known in the art. Suitable methods include but are not limited to transfection, transduction, infection, electroporation, sonoporation, nucleofection, and microinjection. In some embodiments, the nucleic acid construct is introduced into the cell by transfection. Suitable transfection reagents are well known in the art and may include cationic polymers such as polyethylenimine (PEI), cationic lipids such as lipofectamine and related reagents (Invitrogen) and non-liposomal reagents such as Fugene and related reagents (Promega) or Calcium phosphate. In some embodiments, the expression system may be introduced into the cells by transduction using a suitable viral vector such as lentivirus, retrovirus, AAV and adenovirus.

To allow for the selection of cells into which the expression system or a component thereof has been introduced, a selectable marker may be introduced into the cell along with the expression system, or along with one or more expression cassettes of the expression system. The term “selectable marker” as used herein refers to an element in a nucleic acid construct that confers a selective advantage to cells harboring the nucleic acid construct. For example, the selectable marker may encode a protein that is expressed and confers resistance to a specific drug. Alternatively, the selectable marker may encode a protein that is expressed and is essential for cell viability under specific growth conditions. Suitable selectable markers are known to the skilled person. Examples of suitable drug-selectable markers include blasticidin resistance, neomycin resistance, hygromycin resistance, or puromycin resistance.

The selectable marker may be on the same nucleic acid molecule as the expression cassette, or on a different nucleic acid molecule. If the selectable marker is provided on a separate nucleic acid molecule, the nucleic acid molecule with the selectable marker is provided at a lower ratio or percentage than the other nucleic acid molecule(s) e.g. a 1:4 molar ratio, a 1:5 molar ratio, or a 1:10 molar ratio, or e.g. 25%, 20%, or 10% of the total nucleic acids. Cells that take up the selectable marker are likely to have taken up other nucleic acids that are introduced along with the marker, e.g. the expression system or component thereof, thereby allowing for the selection of cells carrying the expression system or component thereof.

A stable cell line comprising one or more expression cassettes of the expression system may be generated by isolating an individual cell comprising the one or more expression cassettes, and culturing the cell to generate a population of cells comprising the one or more expression cassettes. Accordingly, in one embodiment, the method further comprises isolating an individual cell and culturing the individual cell to generate a population of cells.

One or more expression cassettes of the expression system may be introduced into the cell simultaneously and/or sequentially. By way of example, all three expression cassettes may be introduced into the cell in a single step (e.g. a single transfection or single transduction). Alternatively, a first and second expression cassette may be introduced into the cell in a single step, and a third expression cassette, and optionally a fourth and/or fifth expression cassette may be introduced into the cell in one or more subsequent steps. Alternatively, each expression cassette can be introduced into the cell sequentially. Accordingly, in one embodiment, the method of generating a mammalian cell for the production of an RNA or protein of interest comprises introducing into a mammalian cell a first expression cassette of the expression system and a first selectable marker; applying selective pressure to the cell to select for cells that carry the first selectable marker, thereby selecting cells that carry the first expression cassette; isolating a first individual cell comprising the first expression cassette; culturing the first individual cell to obtain a first population of cells comprising the first expression cassette; introducing into a cell of the first population of cells a second expression cassette of the expression system and a second selectable marker; applying selective pressure to the cell to select for cells that carry the second selectable marker, thereby selecting cells that carry the second expression cassette; isolating a second individual cell comprising the second expression cassette; culturing the second individual cell to obtain a second population of cells comprising the second expression cassette; introducing into a cell of the second population of cells a third expression cassette of the expression system and a third selectable marker; applying selective pressure to the cell to select for cells that carry the third selectable marker, thereby selecting cells that carry the third expression cassette; isolating a third individual cell comprising the third expression cassette; and culturing the third individual cell to obtain a third population of cells comprising the third expression cassette.

The mammalian cell comprising the expression system described herein may be used for the inducible production of a complex biologic such as an antibody or a viral vector. Accordingly, in an embodiment, the method further comprises introducing one or more additional expression cassettes encoding additional components into the cell. The additional expression cassettes may be introduced into the cell along with an additional selectable marker and/or along with any other expression cassette and/or any other additional component. For example, where the expression system is used for the inducible production of a viral vector, an additional component may include a viral construct carrying a gene (encoding an RNA or protein) of interest.

The expression systems described herein may be used for the inducible production of one or more RNAs or proteins of interest encoded therein. Accordingly, one aspect of the present disclosure is a method of inducing production of one or more RNAs or proteins of interest, the method comprising: a) obtaining a mammalian cell comprising the expression system of the disclosure encoding an RNA protein of interest; b) adding inducing agents to growth media of the cell to induce expression from the inducible promoters; and c) culturing the cell under conditions for production of the RNA or protein of interest, thereby producing the RNA or protein of interest.

The expression system comprises a cumate-inducible promoter and a coumermycin-inducible promoter. Accordingly, suitable inducing agents include a cumate effector molecule and a coumermycin effector molecule. In one embodiment, the cumate effector molecule is cumate. Any suitable concentration of cumate may be used, for example about 1-200 μg/ml, about 50-150 μg/ml, or optionally about 100 μg/ml. In one embodiment, the coumermycin effector molecule is coumermycin. Any suitable concentration of coumermycin may be used, for example about 1-30 nM, about 5-20 nM, or optionally about 10 nM. The cumate effector molecule and the coumermycin effector molecule can be added to the growth media at about the same time or sequentially.

### IV. COMPOSITIONS OF MATTER

The expression system of the disclosure comprises three or more expression cassettes each comprising a nucleic acid molecule. The expression cassettes described herein may be provided as one or more nucleic acid molecules or constructs. It will be understood that a nucleic acid molecule or construct may be integrated into the genetic material of a cell, or may be incorporated into a plasmid. Accordingly, in an aspect, one or more expression cassettes of the expression system described herein may be provided in the form of one or more plasmids comprising an expression cassette or expression cassettes of the expression system, and/or in the form of a cell comprising one or more expression cassettes of the expression system. In an embodiment, one or more expression cassettes are provided on one or more plasmids. In an embodiment, one or more expression cassettes may be integrated into the genetic material of a cell.

Another aspect of the disclosure includes a mammalian cell useful for the inducible expression of an RNA or protein of interest. Accordingly, in an embodiment, the disclosure provides a mammalian cell comprising the expression system described herein encoding an RNA or protein of interest. As used herein, a “cell comprising an expression cassette”, a “cell comprising the expression system”, or similar phrases, means a cell into which the nucleic acid molecule(s) of the expression cassette or expression system, as indicated, has been introduced. Suitable methods of introducing a nucleic acid molecule into a cell are well known in the art.

In an embodiment, the mammalian cell is useful for the inducible production of a viral vector. Accordingly, in an embodiment, the mammalian cell comprises additional expression cassettes encoding one or more components of a viral vector. In one embodiment, the viral vector is a lentiviral vector. In one embodiment, the viral vector is an adeno-associated virus (AAV).

In an embodiment, the mammalian cell is a viral packaging cell comprising expression cassettes encoding components of a viral vector. In an embodiment the viral packaging cell is a lentiviral packaging cell comprising expression cassettes encoding for example lentiviral REV, Gag/pol, and/or a viral envelope protein such as VSVg, optionally VSVg-Q96H-I57L. In an embodiment, the viral packaging cell is an AAV packaging cell comprising expression cassettes encoding for example Rep 40, Rep 52, Rep 68, Rep 78, or a combination of at least one of Rep 40 or Rep 52, and at least one of Rep 68 or Rep 78. For example, the expression system may comprise Rep 40 and Rep 68, Rep 40 and Rep 78, Rep52 and Rep 68, or Rep 52 and Rep 78.

Viral constructs are made of DNA or RNA and they contain some of the genetic material of the viruses they are derived from (such as lentivirus, retrovirus, AAV and adenoviruses). Viral constructs have been modified to carry and to deliver a gene of interest that will produce a recombinant protein or an RNA of interest and can be used for example for the treatment of diseases by cell and gene therapy and also for vaccination. Viral constructs can also be used to deliver a gene of interest to produce a recombinant protein or an RNA in cell culture. Suitable viral constructs are well known in the art and depend on the type of viral vectors and viruses being used. Accordingly, in an embodiment, the viral packaging cell further comprises a viral construct carrying a gene of interest. The type of viral construct will depend on the viral packaging cell (or the viral vector) being used. For example, where the viral packaging cell is a lentiviral packaging cell, the viral construct is a lentiviral construct. Where the viral packaging cell is an AAV packaging cell, the viral construct is an AAV construct.

In an embodiment, the mammalian cell is useful for the inducible production of an antibody. Accordingly, in an embodiment, the mammalian cell comprises expression cassettes encoding one or more additional components of an antibody.

To provide a flexible tool for customizable expression of one or more RNAs or proteins of interest, the mammalian cell may comprise only a portion of the expression system described herein. For example, the mammalian cell may be an “expression ready” cell comprising a first and second expression cassette of the expression system described herein. The third expression cassette may be incorporated for example into a plasmid which may be customized by a user to encode the RNA or protein of interest, and introduced into the expression ready cell to generate a cell for the inducible production of the RNA or protein of interest.

### V. KITS

The expression system described herein may be provided as a kit for the inducible expression of an RNA or protein of interest. Accordingly, an aspect includes a kit for the inducible expression of an RNA or protein of interest. In one embodiment, the kit comprises plasmids encoding the expression system of the disclosure. In another embodiment, the kit comprises a cell comprising the expression system of the disclosure and one or more inducing agents. In a further embodiment, the kit comprises an “expression ready” cell comprising a first and second expression cassette of the expression system, and a plasmid comprising a coumermycin-inducible promoter, a cloning site, and polyadenylation signal and/or a plasmid comprising a third expression cassette.

Where the expression system produces a viral vector, the kit may comprise a viral packaging cell comprising the expression system of the disclosure encoding components of the viral vector, and a suitable viral construct.

The following non-limiting examples are illustrative of the present disclosure:

### VI. EXAMPLES

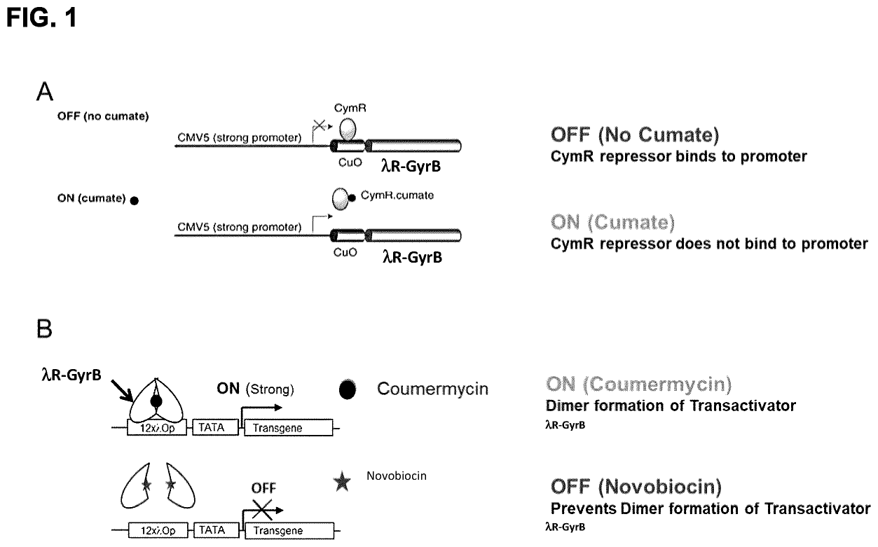

In this disclosure, a cell line, such as one derived from Human Embryonic Kidney cells (HEK293 cells) was engineered to produce the repressor of the cumate gene-switch (known as CymR) and the coumermycin chimeric transactivator (λR-GyrB). The HEK293-derived 293SF-3F6 cells were used herein because they grow in suspension culture and in serum-free medium to facilitate the scale-up and for regulatory compliance. The resulting cell line is referred to as 293SF-CymR/λR-GyrB (FIG. 1).

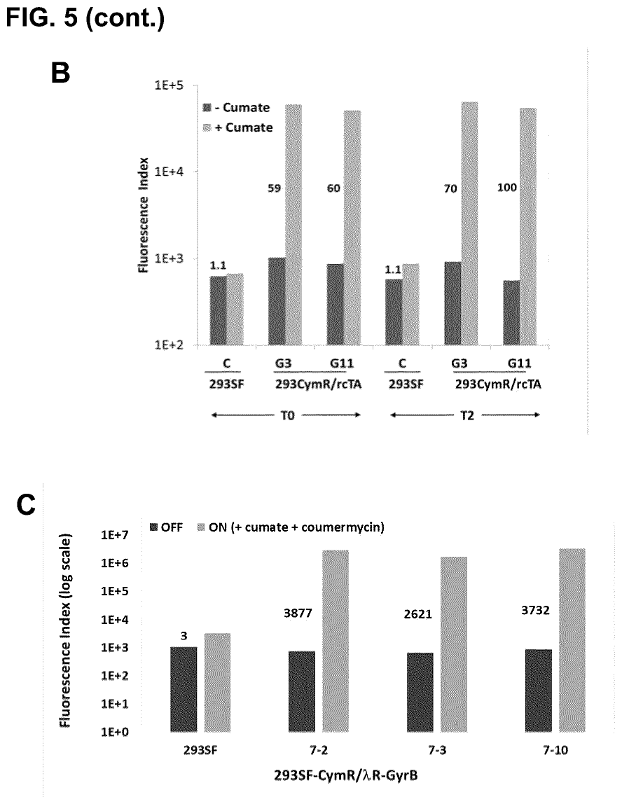

The induction level provided by the cumate/coumermycin gene-switch was 130 fold higher compared to the cumate-gene switch in repressor configuration and 30 fold higher compared to the cumate gene-switch in reverse transactivator configuration, as determined by comparing the On/Off ratio of gene expression before (Off) and after induction (On) (FIG. 5).

The usefulness of a novel cumate/coumermycin gene-switch to manufacture complex biologic drugs by using it to generate packaging and producer cells for viral vectors derived from lentivirus (LV) is demonstrated herein. LV are very important vectors for gene and cell therapy applications (Dropulic, 2011; Escors and Breckpot, 2010; Matrai et al., 2010). The packaging and producer cells for LV described herein are derived from the 293SF-CymR/λR-GyrB cells and are capable of growing in suspension culture and in serum-free medium. To make LV, cells have to produce cytotoxic proteins, such as the envelope glycoprotein of vesicular stomatitis virus (VSVg) and the protease encoded by the Gag/pol gene of human immunodeficiency virus (HIV). Cells containing these genetic elements can be constructed only by using a very tightly regulated inducible gene expression system.

The generation of packaging cells for adeno-associated virus (AAV) is also described herein. AAV is another important viral vector for gene and cell therapy applications (Balakrishnan and Jayandharan, 2014; Kotterman and Schaffer, 2014; Robert et al., 2017; Weitzman and Linden, 2011). More specifically, use of the cumate/coumermycin gene-switch to construct a cell line (293SF-Rep) expressing the highly cytotoxic Rep proteins of AAV is demonstrated herein. Rep proteins are essential for replication and assembly of AAV. As shown herein, 293SF-Rep cells were capable of producing AAV after induction with cumate and coumermycin.

### Example 1. Construction of 293SF-CymR/λR-Gyrb

The first step to generate the 293SF-CymR/λR-GyrB cell line was to construct a stable cell line expressing the repressor of the cumate gene-switch (293SF-CymR).

### Construction of 293SF-CymR Cell Line (Clone 198-2)

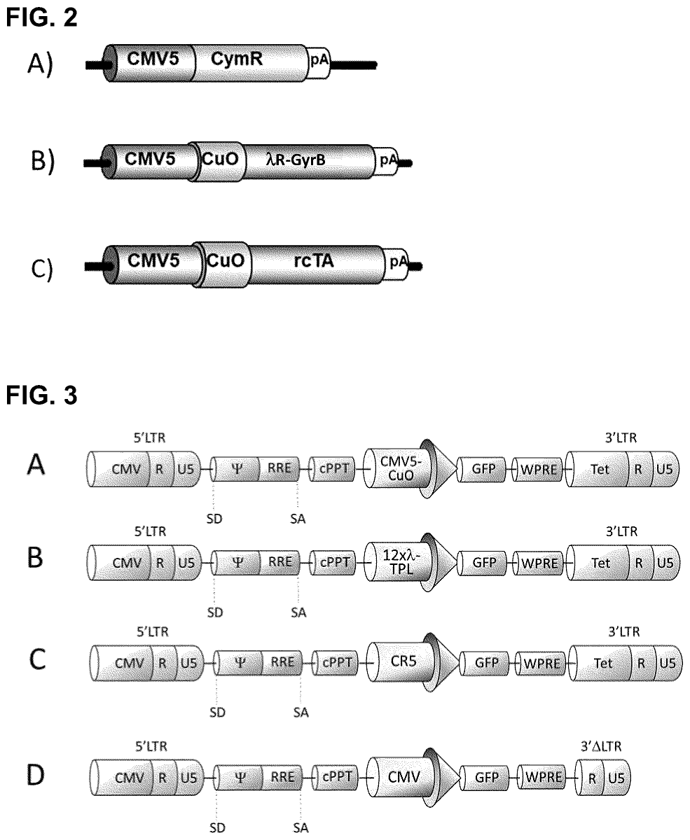

A clone of HEK293 cells that was adapted to serum free suspension culture (clone 293SF-3F6 (Cote et al., 1998)) was used as the recipient for the CymR gene. Briefly, the cells were transfected with a plasmid encoding the CymR gene regulated by the CMV5 promoter (FIG. 2A) (an example of the first expression cassette disclosed herein) and a plasmid encoding the resistance for puromycin. After transfection, the cells were diluted in 96-well plates in the presence of puromycin. Resistant colonies were picked and amplified. The presence of CymR in the clones was tested by transducing them with a LV expressing GFP regulated by the CMV5CuO promoter (LV-CMV5CuO-GFP, FIG. 3A). The level of GFP expression after transduction with LV-CMV5CuO-GFP and induction with cumate was visualized by fluorescence microscopy or it was quantified by flow cytometry by comparing induced and non-induced cells (On/Off ratio). The clones with the best On/Off ratio were expanded and banked (a subset of the cell population that was not induced nor treated with the LV was used for cell expansion and cell banking). The clones with the best On/Off ratio (#169 and #198) were then subcloned by plating them at low cell density in semi-solid medium. Well isolated colonies were then picked using a robotic cell picker (ALS CellCellector™) as described previously (Caron et al., 2009). The subclones were amplified and tested for the capacity of CymR to regulate gene expression by transducing the cells with LV-CMV5CuO-GFP. One of the best subclones is referred to as clone 198-2.

Insertion of λR-GyrB into the 293-CymR Cells

293SF-CymR cells (clone 198-2) were transfected with a plasmid encoding the λR-GyrB transactivator regulated by the CMV5CuO promoter (FIG. 2B) (an example of the second expression cassette disclosed herein) and a plasmid encoding the resistance for blasticidin. After transfection, the cells were plated in 96-well plates in the presence of blasticidin. Resistant colonies were picked, amplified and tested for the presence of λR-GyrB by transducing them with a LV expressing GFP regulated by the 12xlambda-TPL promoter (LV-12xlambda-TPL-GFP, FIG. 3B). The best clones were selected based on the highest On/Off ratio by measuring GFP expression by flow cytometry after induction with cumate and coumermycin. The best clones were amplified and banked as described above. They were subcloned by limiting dilution in 96-well plates. Colonies were picked, expanded and tested for the presence of λR-GyrB as described above by transduction with LV-12xlambda-TPL-GFP and analysis by flow cytometry after induction.

The Cumate/Coumermycin Gene-Switch Provides a Better Induction Level than the Cumate Gene-Switch

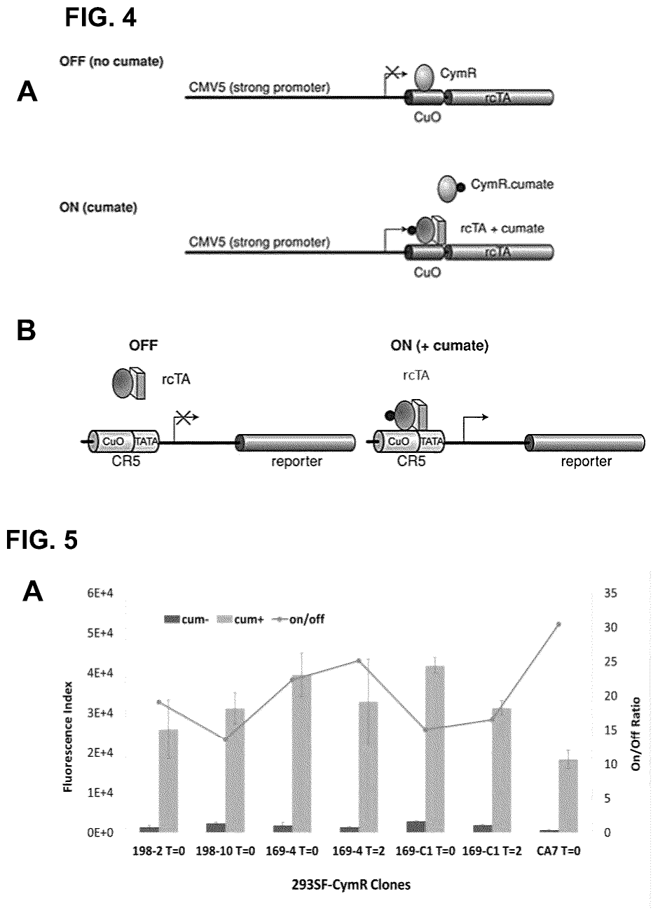

The efficacy of the cumate/coumermycin gene-switch was evaluated by testing the On/Off ratio of three of the best clones of 293SF-CymR/λR-GyrB after transduction with LV-12xlambda-TPL-GFP. The On/Off ratio obtained with the best clones of 293SF-CymR (described above) and the best clones of 293SF-CymR/rcTA was also tested for comparison. Clones of 293SF-CymR/rcTA were generated by transfecting 293SF-3F6 cells with plasmid encoding CymR regulated by CMV and a plasmid encoding the reverse transactivator rcTA regulated by CMV5CuO promoter (FIG. 2C) and by isolating clones that have stably integrated both genes into their chromosomes as described above. In the case of 293SF-CymR/rcTA, addition of cumate activates the transcription of the rcTA gene (by releasing the inhibition by CymR). After synthesis the rcTA binds to the CR5 promoter to activate transcription (Mullick et al., 2006) (FIG. 4). A detailed description of the steps used to construct the 293 SF-CymR/rcTA clones is provided in the section entitled materials and methods.

The On/Off ratios of 293SF-CymR and 293SF-CymR/rcTA clones were tested by transducing the cells with LV-CMV5CuO-GFP and LV-CR5-GFP respectively and by measuring the GFP expression level by flow cytometry after induction. LV-CR5-GFP carries a GFP gene regulated by the CR5 promoter (FIG. 3C). The On/Off ratios observed were approximatively 30, 100 and 4000 for the 293SF-CymR, 293SF-CymR/rcTA and 293SF-CymR/λR-GyrB clones respectively (FIG. 5). The fact that the On/Off ratio was 130 (4000/30) and 40 (4000/100) fold higher for the cells expressing CymR/λR-GyrB in comparison to the cells expressing only CymR or cells expressing the CymR/rcTA combination, indicates that the cumate/coumermycin switch provides a much better induction level.

### Example 2. Construction of Packaging Cells for Production of LV

As one example to demonstrate the usefulness of the cumate/coumermycin gene-switch to produce complex biologic products, one of the 293SF-CymR/λR-GyrB clones was used to construct an inducible packaging cell line for the production of LV. LV are of paramount importance for cell therapy because they are used to genetically modify cells that are delivered to patients to treat cancer or genetic disorders (Dropulic, 2011; Milone and O'Doherty, 2018). One of the challenges in the field of cell therapy is to produce at a reasonable cost and in a timely manner, the required amount of good quality LVs for clinical application.

One solution to facilitate the production of LV is to construct packaging cells that contain all of the genetic elements necessary for the assembly of LV. In the case of third generation LV, three genes are necessary to produce LV: Rev, Gag/Pol and the envelope protein (Cockrell and Kafri, 2007; Dropulic, 2011; Pluta and Kacprzak, 2009). The most common envelope protein used is VSVg. With the availability of a packaging cell line, scientists can generate stable producer clones that can produce LV without the need of transfection. The advantage of producer clones over transient transfection is reproducibility and simplicity (no transfection and no preparation of plasmid). The utilisation of packaging cells adapted to suspension culture will greatly facilitate the scale-up of LV production. In addition, the use of serum-free culture media will lead to a product that is safer and better characterized, thus facilitating cGMP manufacturing.

Some of the genes (Gag/pol and VSVg) needed to produce LV are cytotoxic and therefore they must be tightly turned off during cell growth and cell banking to avoid killing the host cells. For this reason, the successful construction of packaging cells for LV has been possible only by using an efficient inducible expression system (Broussau et al., 2008; Farson et al., 2001; Kafri et al., 1999; Ni et al., 2005; Pacchia et al., 2001; Sanber et al., 2015; Sparacio et al., 2001).

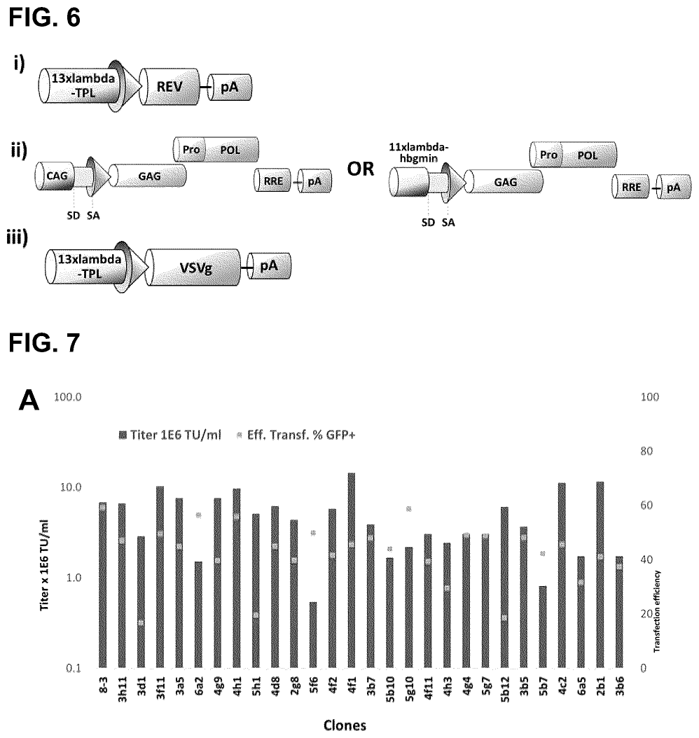

The first step to generate a packaging cell was to construct plasmids carrying the Rev, Gag/pol and VSVg genes. First, plasmids were constructed in which the transcription of Rev and VSVg is controlled by the 13xlambda-TPL promoter (FIGS. 6 and 15). Two different promoters were tested for expression of Gag/Pol: 11xlambda-hbgmin and the CAG promoter (FIGS. 6 and 16). CAG is a strong constitutive hybrid promoter made by the fusion of the CMV enhancer to the actin promoter (Miyazaki et al., 1989). In the latter case, despite the fact the Gag/pol is regulated by the strong constitutive CAG promoter, its mRNA cannot be transported to the cytoplasm and translated into a polyprotein in the absence of the Rev protein. For this reason, the presence of Rev is needed for the production of Gag/pol polypeptide. The transcription of the Rev gene, therefore indirectly controls the synthesis of the Gag/pol polypeptide.

Two strategies were employed to produce packaging cells. In the first one, 293SF-CymR/λR-GyrB cells were transfected with plasmids for 11xlambda-hbg-Gag/Pol, 13xlambda-TPL-Rev, 13xlambda-TPL-VSVg (examples of the third, fourth, and fifth expression cassettes as disclosed herein) and with a fourth plasmid encoding resistance to neomycin (an example of a selectable marker as disclosed herein). In the second strategy, 293SF-CymR/λR-GyrB cells were transfected with plasmids for CAG-Gag/Pol, 13xlambda-TPL-Rev, 13xlambda-TPL-VSVg and a plasmid encoding resistance to hygromycin (another example of a selectable marker as disclosed herein).

After transfection, the selective agent was added to the cell culture medium. The pool of resistant cells was cloned by dilution into nanowells. Resistant colonies derived from a single cell (as documented by taking a picture at the time of plating) were isolated using a robotic cell picker (CellCelector™) and transferred into 384 well-plates. The cells were then expanded and tested for the production of LV.

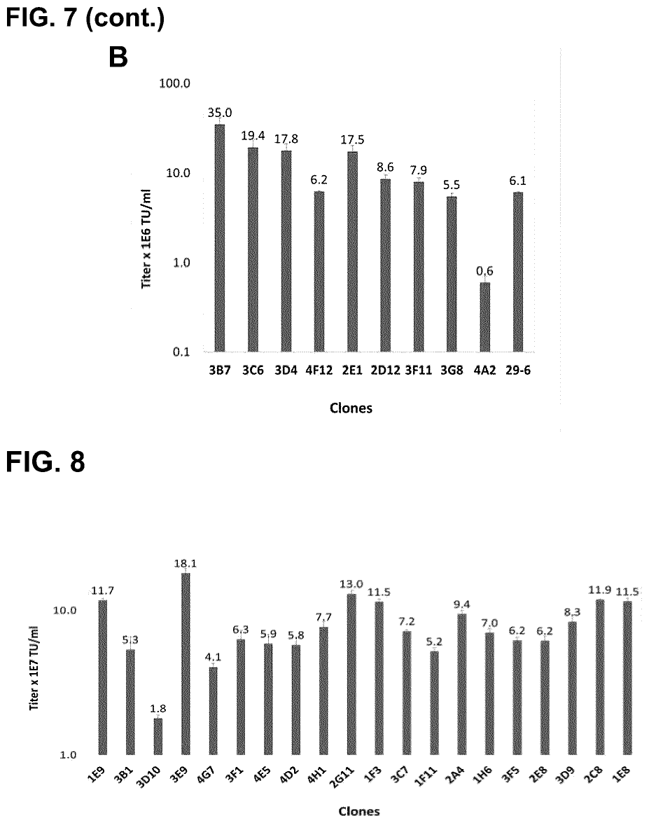

The clones for the packaging cells were screened for the production of LV by transfecting them with a plasmid encoding a LV expressing GFP regulated by CMV (LV-CMV-GFP) (FIG. 3D) (an example of a lentiviral construct disclosed herein). A subpopulation of cells that were not transfected was kept aside for amplification of the best clones and cell banking. The titers of the LV-CMV-GFP produced after transient transfection was measured by flow cytometry after transduction of HEK293 cells. Both strategies, using either CAG-Gag/Pol plasmid or 11xlambda-hbgmin-Gag/Pol plasmid, were capable of generating packaging cells with titers above 1.0×10⁶ Transducing units (TU) per ml in the culture medium. Several clones also produced titers above 1.0×10⁷ TU/ml. (FIG. 7). Previous attempts at isolating clones producing LV using packaging cells constructed with the cumate-switch only were unsuccessful (not shown).

### Example 3. Construction of Stable Producers for LV

Packaging cells (Clone 3D4 (FIG. 7B)), were used to generate producer clones with the capacity to make LV without the need of transient transfection. Briefly, the packaging cells 3D4 were co-transfected with a plasmid encoding LV-CMV-GFP (FIG. 3D) and a plasmid encoding the resistance for neomycin. After transfection, the selective agent (neomycin) was added to the cells and the neomycin resistant colonies were cloned by dilution into nanowell plates. Colonies were isolated using a robotic cell picker (CellCelector™) and transferred into 384 well plates. The clones were expanded and tested for the production of LV-CMV-GFP by adding the inducers (cumate and coumermycin). The LV was titrated by flow cytometry following transduction of HEK293 cells. Several clones were able to produce LV-CMV-GFP in the range of 1.0×10⁸ TU/ml in the culture medium (FIG. 8).

### Regulation of Gene Expression in the Packaging Cells

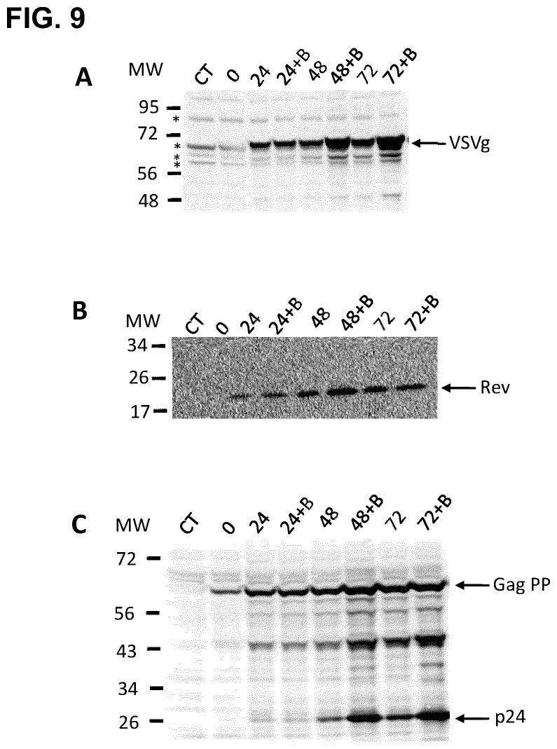

To confirm the efficacy of the cumate/coumermycin gene-switch in the context of the packaging cells for LV, the expression of the genetic elements (Rev, Gag and VSVg) necessary to produce LV was analysed by western blot before and after induction. As expected, expression of Rev, Gag, and VSVg was strongly induced after addition of cumate and coumermycin (FIG. 9) and very weak or no expression was detected before induction.

### Example 4. Construction of Packaging Cells for the Production of AAV

One popular method to produce viral vectors derived from adeno associated virus (AAV) is by transient transfection of HEK293 cells with three plasmids carrying the elements necessary to assemble functional AAV particles (Balakrishnan and Jayandharan, 2014; Grieger and Samulski, 2012; Robert et al., 2017; Wright, 2009). The plasmids are: i) the expression plasmid that carries the gene to be delivered by the AAV (the viral construct), ii) the helper plasmid that encodes essential helper genes derived from adenovirus and iii) the Rep-Cap plasmid that contains the Rep and Cap genes of AAV. Rep produces four proteins (Rep40, Rep52, Rep68 and Rep78) involved in the replication and packaging of the AAV genome. Cap encodes the structural proteins making up the capsid of the AAV. One potential approach to facilitate the production of AAV would be to use packaging cells that contain, as in the case of LV, the elements needed to produce AAV. However, it is difficult to generate packaging cells for AAV because Rep codes for cytotoxic proteins. As an additional proof for the usefulness and efficacy of the cumate/coumermycin gene-switch, packaging cells for AAV (293SF-Rep) were constructed that produce the Rep proteins under the control of this gene-switch. Use of the 293SF-Rep cells to produce AAV was also demonstrated.

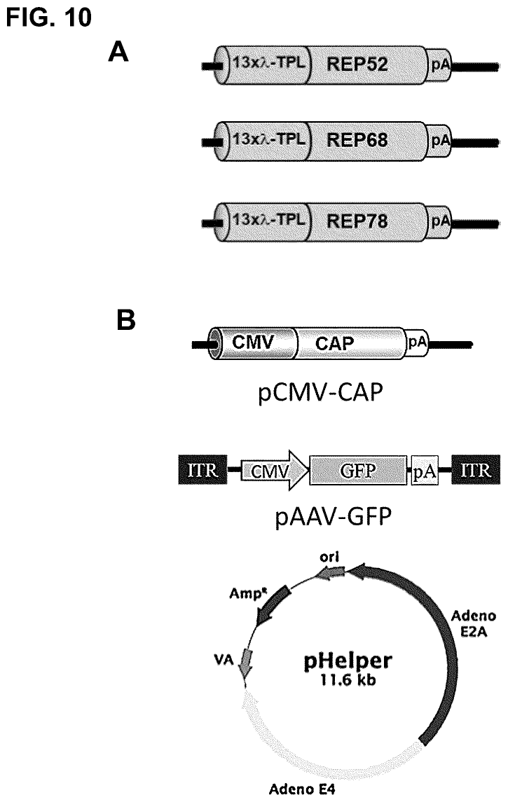

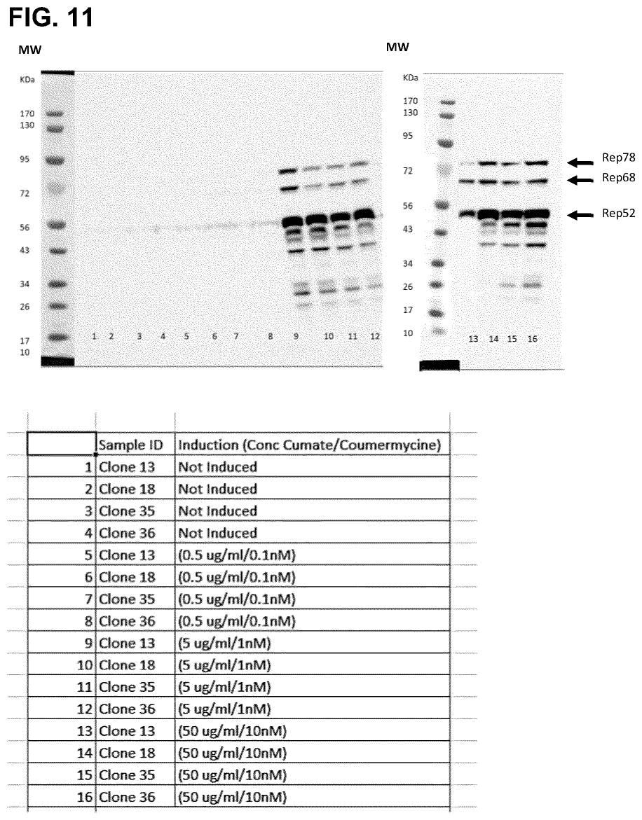

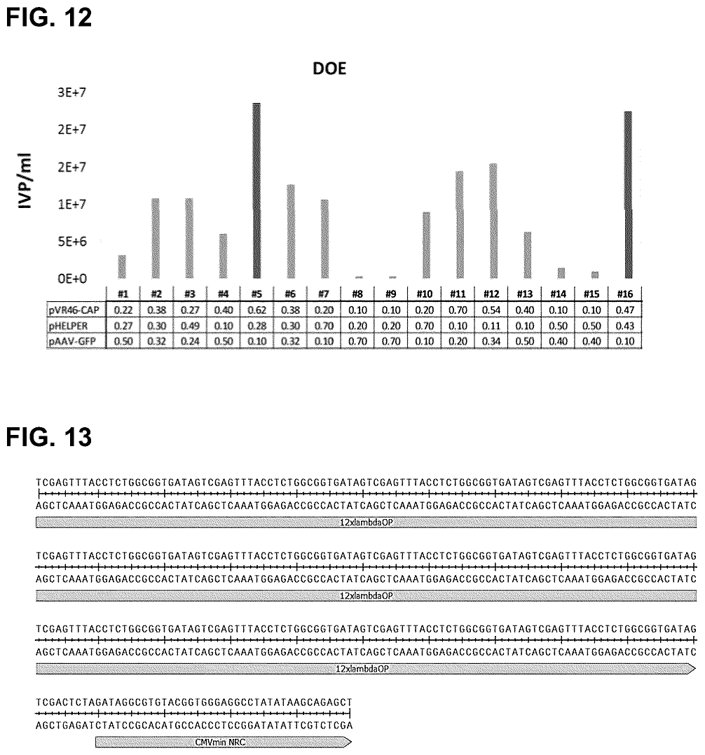

To construct 293SF-Rep cells, 293SF-CymR/λR-GyrB cells were transfected with a plasmid encoding Rep52, a plasmid encoding Rep68 and a plasmid encoding Rep78, each regulated by 13xlambda-TPL promoter (FIG. 10A) (examples of a third, fourth, and fifth expression cassette disclosed herein), and a plasmid encoding the resistance for hygromycin (an example of a selectable marker as disclosed herein). For this experiment, a plasmid for Rep40 was not included because Rep40 is not essential to produce AAV in the presence of Rep52 (Chahal et al., 2018). After transfection, hygromycin was added to the culture medium and a hygromycin resistant pool was generated and then cloned by limiting dilution in 96-well plates. Hygromycin resistant colonies were isolated expanded and tested for the production of AAV by transient transfection with three plasmids: for Cap (pCMV-CAP), for the adenovirus helper genes (pHelper), and for the expression plasmid carrying GFP (pAAV-CMV-GFP) regulated by the CMV promoter (FIG. 10b). The quantity of AAV produced was measured by transducing HEK293A cells with the AAV and scoring the percentage of GFP positive cells semi-quantitatively using a fluorescence microscope, or quantitatively by flow cytometry. The clones with the capability to produce AAV were amplified and banked (a subpopulation of cells that were not transfected nor induced were used for this purpose). The production of Rep proteins, following induction with cumate and coumermycin was demonstrated by western blot for some of these clones (FIG. 11). The production of AAV from clone 13 was investigated using different ratio of plasmids (FIG. 12). Under some conditions, clone 13 was able to produce 2.5×10⁷ infectious virus particles (IVP) of AAV-CMV-GFP per ml by transient transfection. In the absence of induction, the amount of AAV produced was below the sensitivity of the method.

### Materials and Methods

### Plasmid Construction

Plasmids were constructed using standard methods of molecular biology and they were purified by chromatography using commercial kits (Qiagen Valencia, CA) after amplification in E. coli. After purification, the plasmid concentration was measured at 260 nm using the NanoDrop™ spectrophotometer (Thermo Scientific). Plasmid integrity was confirmed by digestions with restriction enzymes. The plasmids needed for this project were generated as described below.

pBlast: the sequence for the expression cassette for the gene for the resistance for blasticidin cloned into pUC57 was ordered from a gene synthesis company (GenScript).

pHygro: the sequence for the expression cassette for the resistance for hygromycin cloned into pUC57 was ordered from a gene synthesis company (GenScript)

pkCMV5-CuO-mcs; This plasmid was made by removing the Rev gene from pkCMV5-CuO-Rev (Broussau et al., 2008) by digesting with restriction enzymes.

pKCMV5-CuO-rcTA-Hygro: the rcTA sequence (Mullick et al., 2006) was removed from pAdenovatorCMV5-CuO-rcTA by BlpI and SwaI digestions and used to replace the Rev Sequence of pKCMV5-CuO-Rev (Broussau et al., 2008) by digestion with KpnI (blunted) and BlpI, thus generating the pKCMV5-CuO-rcTA plasmid. The hygromycin expression cassette was removed from pMPG-CMV5-CymRopt-Hygro (Gilbert et al., 2014) by digestion with NruI and BssHII, the ends were filled-in and ligated into pKCMV5-CuO-rcTA plasmid previously digested with AflII and filled-in.

pKCMV5-CuO-λR-GyrB: the λR-GyrB sequence was removed from pGyrb (Zhao et al., 2003) by NdeI and DraI digestion and the ends were filled-in. The DNA fragment containing the λR-GyrB sequence was used to replace the Rev Sequence of pKCMV5-CuO-Rev (Broussau et al., 2008).

pLVR2-CR5-GFP: the LV vector sequences (RSV to GFP) from the pRRL.cppt.CR5-GFP.WPRE (Mullick et al., 2006) was transferred into the LVR2-GFP plasmid (Vigna et al., 2002) by digestion with SphI and SalI.

9_SG_pMA-12xlambda-CMVmin-Protease: This plasmid was ordered from GeneArt (ThermoFisher). It contains the adenovirus protease sequence under the control of the 12xlambda-CMVmin promoter.

pME_005 (pVV-13xlambda-CMVmin-VSVg-Q96-157L): The CMV5 promoter of pNN02 was replaced with the 12xlambda-CMVmin fragment amplified from pVR10 (see below). Sequencing revealed that the resulting plasmid had 13 copies of the lambdaOp instead of 12 copies.

pMPG-CMV5-CymR: the Hygromycin expression cassette was removed from pMPG-CMV5-CymRopt-Hygro (Gilbert et al., 2014) by NruI/AscI (filled-in) digestions and ligated to re-circularise the plasmid.

pMPG-Puro: the puromycin expression cassette was isolated from plasmid pTT54 (Poulain et al., 2017) and inserted into plasmid derived from pMPG (Gervais et al., 1998) that did not contain an insert.

pNN02 (pVV-CMV5-VSVg-Q96-157L): To construct this plasmid, we first made pVV-CMV5 by removing a BglII/BbsI fragment containing the DS and FR sequence from pTT5 vector (Durocher et al., 2002), filling-in the ends and re-circularizing the plasmid. The VSVg gene (VSVg-Q96-157L shown in SEQ ID NO: 19, which is codon optimized based on the amino acid gene bank accession number ABD73123.1 shown in SEQ ID NO: 20) ordered from GenScript was then cloned into PmeI site of pVV-CMV5.

pNRC-LV1 (pNC109): Three DNA fragments containing the complete backbone of a lentiviral vector (See FIG. 3, without expression cassette [CMV-GFP]) were ordered from a gene synthesis company (Integrated DNA Technologies) and then combined by Gibson assembly into a cloning vector derived from pMK (GeneArt™, ThermoFisher). The resulting plasmid is referred to as pBV3. To obtain a higher titer, a fragment (CMV5‘UTR-HIV-l’F-RRE-cPPT, Genbank accession number FR822201.1) was ordered from a gene synthesis company (GeneScript) and used to replace the homologous region in pBV3 by XbaI/SalI digestion.

pNRC-LV1-CMV-GFPq (pNC111): the CMV-GFP was amplified by PCR from pCSII-CMV-GFPq (Broussau et al., 2008) and introduced by Golden Gate assembly into pNRC-LV1 with Esp3I sites.

pSB178 (pKCR5-VSVg-Q96H-I57L) and pSB174 (pKCR5-Rev) are both derived from pKCMV-B43 vector (Mercille et al., 1999) that was modified to replace the CMV-B43 cassette with the CR5 promoter from the cumate switch (Mullick et al., 2006). The sequences of VSVg-Q96H-I57L (SEQ ID NO: 19, which is codon optimized based on the amino acid gene bank accession number ABD73123.1 as shown in SEQ ID NO: 20) and Rev (Gene bank accession number AF033819.3) were ordered from a gene synthesis company (GenScript) and cloned downstream of the CR5 promoter to make pSB178 and pSB174 respectively.

pSB189 (pkCMV5-hbgdelta-Gag/pol2): the Gal/pol gene (HIV-1 complete genome Gene bank accession number AF033819.3) and a portion of the intron of human beta globin (gene bank accession MK476503.1) were ordered from GenScript. Both fragments were cloned into a plasmid derived from pKCMV-B43 (Mercille et al., 1999) that was modified by replacing the CMV-B43 cassette with the CMV5 promoter (Massie et al., 1998a). During the cloning, part of the intron of CMV5 was replaced by the human beta-globin intron sequence.

pSB201 (pMPG/TK*/Neo): the plasmid was generated by removing the CymR-nls cassette from pMPG/TK*neo/CymR-nls (Mullick et al., 2006) by AscI digestion.

pSB211 (pCAG-Gag/polIIIb): the Gag/PolIIb sequence (GeneBank accession number EU541617.1) was ordered from a gene synthesis company (Genscript) and cloned into a plasmid derived from pKCMV-B43 (Mercille et al., 1999) that was modified by replacing the CMV-B43 cassette with the CAG promoter (Blain et al., 2010).

pSB213 (p11xlambda-hbgmin-Gag/polIIIb): the 12lambda-hbgmin promoter and intron was extracted from pVR28 by digestion with BamHI (filled in) and used to replace the CAG promoter of pSB211 (pCAG-Gag/polIIIb) by digestion with XhoI (filled) and EcoRI (filled). After sequencing the promoter, it appeared that one lambdaOp repeat was lost during cloning and that 11 lambdaOp repeats (instead of 12) were left in the promoter. A schematic of the resulting promoter (11xlambda-hbgmin) is shown in FIG. 16.

pTet07-CMV5-CuO-GFP: the CMV5-CuO sequence was first extracted from pRRL.cppt.CMV5-CuO-rcTA (Mullick et al., 2006), by digestion with SpeI and BamHI, the ends were filled in and ligated into pNEB193mcs (NEB) previously digested with XbaI and blunted thus generating pNEB-CMV5-CuO. The CMV5-CuO sequence from pNEB-CMV5-CuO was next ligated into pTet07-CSII-5-GFP (described below) after digestion of both DNAs with PacI and BlpI.

pTet07-CSII-CMV-mcs: the plasmid LVR2-GFP (Vigna et al., 2002) was first modified by inserting a BspEI linker (TCGATCCGCA) in the XhoI site. The 3′LTR containing the Tet07 operator was then removed from this construct by digestion with BspEI and PmeI and ligated into pCSII-CMV-mcs (Miyoshi et al., 1998) previously digested with BspEI and BsmI (previously blunted).

pTet07-CSII-mcs: The CR5 promoter of pTet07-CSII-5-mcs was removed by digestion with PacI and AgeI to generate an empty LV backbone.

pTet07-CSII-5-mcs and pTet07-CSII-5-GFP: the CMV promoter from Tet07-CSII-CMV-mcs and Tet07-CSII-CMV-GFP (Broussau et al., 2008) was replaced with the CR5 promoter and a PacI site was inserted at the 5′ end of the promoter to easily change the promoter in future constructs. Briefly, we first amplified a PCR fragment that covers a portion from a SnabI site in the first CMV promoter (in the 5′ end of the 5′LTR) to the cppt sequence using a plasmid derived from pCSII-CMV mcs (Miyoshi et al., 1998) as template. A second PCR was performed to amplify the CR5 promoter from pRRL.cppt.CR5-GFP.WPRE (Mullick et al., 2006). A PacI site was included at the 3′ end of the first fragment and at the 5′ of the second. The PCR products were annealed and treated with T4 DNA polymerase to generate one fragment covering together the portion from the CMV in 5′ of the 5′LTR to the mcs. This fragment was next inserted into Tet07-CSII-CMV-mcs and Tet07-CSII-CMV-GFP plasmids by digestion with SnabI and AgeI. Sequencing revealed that 5 of the 6 CuO copies of the CR5 promoter had been deleted during the cloning.

pTet07-CSII-12xlambda-TPL-GFPq: the 12xlambda-TPL-GFPq sequence from pVR9 was first inserted into pUC19 by digestion with BamHI thus generating pUC19-12xlambda-TPL-GFPq. The 12xlambda-TPL-GFPq sequence was next ligated into Tet07-CSII-mcs after digestion with EcoRI.

pVR1 (pKC_12xlambda-CuO-TPL-MCs): The 12xlambda-CuO promoter was ordered form GenScript and used to replace the CMV-CuO promoter region of pKCMV5-CuO-MSC by digestion with KpnI/AgeI

pVR2: (pKC_12xlambda-CuO-msc-PolyA) was generated by introducing 12xlambda-TATA-CuO synthesized by GenScript into Acc65I/BglII sites of pKCMV5-CuO-mcs in place of CMV5-CuO promoter.

pVR5 (pKC_12xlambda-CuO-TPL-GFPq): The GFP gene, obtained by digesting pAd-CMV5-GFPq (Massie et al., 1998b) with BamHI, was ligated to pVR1 digested with BglII.

pVR6 (pKC_12xlambda-CuO-GFPq): was obtained by subcloning GFPq from pAdCMV5-GFPq (Massie et al., 1998b) digested with BamHI into BglII-digested pVR2.

pVR9 (pKC_12xlambda-TPL-GFPq): the 12xlambda-CMVmin promoter of 9_SG_pMA-12xlambda-CMVmin-Protease was amplified by PCR and was inserted into pVR5, previously digested with AgeI/KpnI, thus replacing the 12xlambda-CuO fragment of pVR5.

pVR10 (pKC_12xlambda-CMVmin-GFP): was made by replacing the KpnI-AgeI fragment (containing 12xlambda-CuO) of pVR6 with similarly-digested 12xlambda-CMVmin fragment, previously amplified by PCR using 9_SG_pMA-12xlambda-CMVmin-Protease as a template.

pVR17 (pVV-13xlambda-CMVmin-MCS): was obtained by removing the CMVmin-VSVg fragment from pME_005 by digestion with SalI and HindIII and by replacing it with a DNA fragment containing CMVmin promoter obtained by PCR using plasmid pVR10 as template.

pVR19 (pVV-13xlambda-TPL-VSVg-Q96H_I57L): was obtained by subcloning a SacI/HindIII fragment containing the cDNA of VSVg (TPL-VSVg) from pSB178 into similarly digested pVR17 (pVV-13lambda-CMVmin-mcs).

pVR21 (pVV-13xlambda-TPL-Rev): was obtained by subcloning the Rev sequence (TPL-Rev) from pSB174 into pVR17 using StuI/NheI restriction sites.

pVR28 (pK-12xlambda-hbgmin_ex_Gag-Pol): a AflIII/XhoI fragment of pSB189 encoding the CMV promoter/enhancer and TPL was replaced by an AflIII/XhoI fragment containing the 12xlambda promoter and TPL from pVR9.

pVR41, pVR42, pVR43 and pVR44: plasmids that contain the AAV2 genes encoding Rep78, Rep68, Rep52 and Rep40, respectively, placed under the control of the 13xlambda-TPL promoter. The plasmids were generated as follows: the human-optimized AAV2 genes encoding Rep78, Rep68, Rep52 and Rep40 were synthesized by GenScript and each was subcloned into the EcoRV site of pUC57 giving rise to 18_SG, 19_SG, 20_SG and 21_SG, respectively. The TATA box of the internal P19 promoter within the Rep 68 and Rep 78 sequences was modified to reduce its activity by changing the TATTTAAGC sequence to the TACCTCTCA sequence. pUC57-Rep clones were digested with BglII/NotI and the fragments encoding the Rep genes were subcloned into similarly-digested pVR19 (pVV-13xlambda-TPL-VSVg) in place of VSVg to give rise to pVR41 (pVV-13xlambda-TPL-Rep78), pVR42 (pVV-13xlambda-TPL-Rep68), pVR43 (pVV-13xlambda-TPL-Rep52) and pVR44 (pVV-13xlambda-TPL-Rep40).

pVR46-Cap: encodes the CAP gene of AAV2 regulated by the CMV5 promoter. To construct this plasmid, the CAP gene of AAV2 and its upstream untranslated region was cloned after the CMV5 promoter (Massie et al., 1998a) of pTT3 thus generation pTT3CAP1. The splice acceptor site of the CMV5 promoter and the splice donor site of the Cap gene were then removed by digesting with AleI/SwaI and by ligating the ends.

### Cell Culture

293SF-CymR and 293SF-CymR/rcTA were cultured in SFM4-Transfx-293 medium (Hyclone) supplemented with 6 mM L-glutamine (Hyclone). Sub-cloning in semi-solid media was performed with a mixture of ClonaCell™ FLEX methylcellulose (StemCell Technology), 2× SFM4-Transfx-293 (Hyclone), 6 mM L-glutamine, 2.5% ClonaCell™ ACF CHO supplements (StemCell Technology). 293SF-CymR/λR-GyrB was developed in Low-Calcium-SFM media (LC-SFM) (Gibco), supplemented with 6 mM L-glutamine and 10 mg/ml rTransferin (Biogems) and expanded in suspension in SFM4-Transfx-293. Cell lines 293SF-PacLVIIIB-L and 293SF-LVPIIIB-GFP were developed in a mixture of 50% LC-SFM supplemented with 6 mM L-glutamine and 10 mg/ml rTransferin and 50% Hycell™ TransFx-H (Hyclone) supplemented with 4 mM L-glutamine and 0.1% Kolliphor® and maintained in suspension in 100% Hycell™ TransFx-H. For suspension culture, the cells were grown in shake flasks at 110 rpm. The 293A (American Type Culture Collection), 293rtTA (Broussau et al., 2008) and 293rcTA (Mullick et al., 2006) were grown in Dulbecco's modified Eagle's medium (Hyclone) supplemented with 5% fetal bovine serum (Hyclone). All cell lines were maintained at 37° C. in a 5% CO₂ humidified atmosphere.

### Production and Titration of Lentivirus

LV-CMV5CuO-GFP, LV-12xlambda-TPL-GFP and LV-CR5-GFP were produced using the 293SF-PacLV #29-6 as described previously (Broussau et al., 2008) by transient transfection with pTet07-CSII-CMV5-CuO-GFP, pTet07-CSII-12xlambda-TPL-GFP and pLVR2-CR5-GFP respectively, in static condition in LC-SMF+1% FBS and addition of 1 μg/mL of doxycycline and 50 μg/mL cumate. Produced LV was concentrated by ultracentrifugation on a sucrose cushion (Gilbert et al., 2007) and the suspensions were used to transduce fresh 293SF-PacLV #29-6 to generate pools of producer cell lines for each LV. The pools were amplified in suspension in LC-SFM+1% FBS and LV production was induced with the addition of 1 μg/mL of doxycycline and 50 μg/mL cumate. Produced LVs were harvested at 48 h and 72 h, concentrated by ultracentrifugation on a sucrose cushion and frozen at −80° C. The LVs CMV5CuO-GFP, 12xlambda-TPL-GFP and CR5-GFP were titrated by transducing 293A, 293rtTA and 293rcTA cells respectively and percentage of GFP expressing cells was analyzed by flow cytometry as described previously (Broussau et al., 2008)

### Generation of Cell Lines

### 293SF-CymR

The 293SF-3F6 cell line (Cote et al., 1998) grown in SFM4-TransFx-293 was transfected using Lipofectamine™ 2000 CD (Invitrogen) with pMPG-CMV5-CymR-opt and pMPG-Puro using a DNA ratio of 9:1. Both DNAs were previously digested with MfeI. 48 hours later the cells were diluted in 96 well plates at 5 000 and 10 000 cells/wells in SFM4-Transfx-293 media containing 0.4 μg/ml of puromycin (Sigma). Selected cell clones were sub-cloned by plating the cells in semi-solid media at 1000 cells/ml and 3000 cells/ml. Colonies were isolated using the CellCelector™, a robotic cell picker (ALS, Germany) and transferred into a 96 well plate. To screen the clones for the presence of CymR, each clone was split into two populations. One population was used for analysis of GFP expression, whereas the other one remained untouched for clone expansion and banking. Clones were analyzed for GFP expression following transduction with a LV expressing GFP regulated by the CMV5CuO promoter (LV-CMV5CuO-GFP). Transductions were first performed in 96 well plates and cells were induced with cumate (Sigma-Aldrich) at 100 μg/ml. Clones were selected for GFP intensity and a second screen was next performed for GFP expression under On/Off conditions (with and without inducers).

### 293SF-CymR-rcTA

293SF-3F6 cells were transfected using Lipofectamine™ 2000 CD with pMPG-CMV5-CymR-opt and pMPG-Puro plasmids (ratio 9:1). Both DNAs were previously digested with MfeI. Two days later, the cells were diluted at 5000 and 10 000 cells per well in 96-well plates, in the presence of 0.4 μg/ml puromycin. The puromycin resistant colonies were transferred into 48- or 24-well plates. When the cells reached a confluency between 50 to 80%, they were combined together in 25 cm² flasks to form 5 different pools (C, D, E, F, G). From this point forward, no more puromycin was added to the medium. Pools were mixed together to form the pool CDEFG.

The pool CDEFG were transfected with PEIpro® (Polyplus Transfection) with a 1:1 complex of linearized (XmnI) pkCMV5-CuO-rcTA-Hygro plasmid. After 24 h of incubation, the cells were transferred in medium containing 25 μg/mL of Hygromycin B, (Invitrogen) in 96-well plates using 1500 cells per well. Colonies were pooled, centrifuged and resuspended in fresh medium containing 25 μg/mL of Hygromycin to form different mini-pools (letters A to H).

The pools C, F, G and H were plated as fully dispersed cells in semi-solid medium. Colonies in semi-solid medium were screened for the presence and level of rcTA by the automated TiSSM method (Transfection in Semi-Solid Medium) and positives colonies were isolated in 96 well plate by the CellCelector™. Briefly, colonies were identified by scanning using the CellCelector™. A 3:1 complex of PEI MAX® (Polysciences) and reporter plasmid encoding the DsRed fluorescent protein (Clontech laboratories) under the control of the CR5 promoter (pkCR5.DsRed) was deposited on each colony with the CellCelector™ robotic arm. At 24 hours post transfection, a scan was performed to detect basic DsRed fluorescence from the colonies (OFF level). A liquid SFM4 medium overlay containing the Cumate inducer (Sigma-Aldrich, Cat No. 268402, Lot No. 13613HB), at 100 g/mL final concentration, was then applied on the semi-solid medium layer to diffuse overnight. At 48 hours post transfection (24 h post induction), a second scan was performed to measure the DsRed fluorescence (ON level). The OFF and ON images were then compared to identify the colonies with high induction characteristics, and these colonies were picked and deposited in individual wells (96-well plate) by the CellCelector™. Clones isolated from TiSSM were gradually transferred to 24-well plates and into 25 cm² flasks and then banked.

### 293SF-CymR/λR-GyrB

293SF-CymR (clone 198-2) was transfected by PEIpro® with plasmids pKCMV5-CuO-λR-GyrB and pBlast at a DNA ratio of 9:1. DNAs were previously digested with XmnI and XbaI respectively. 48 hours post-transfection, the cells were diluted with LC-SFM medium containing 7 μg/ml of blasticidin (Enzo) and were transferred into 96-well plates at 1000 cells/well. After one week, blasticidin concentration was increased at 10 μg/ml. Selected clones were sub-cloned by limiting dilutions in 96 wells at 0.3 cells/well and 1.0 cells/well in LC-SFM media without selection. To screen the clone for their capacity to regulate gene expression, each clone was split into two populations. One population was used for analysis of GFP expression, whereas the other one remained unmodified for clone expansion and banking. Clones were analyzed for GFP expression following transduction with a lentiviral vector expressing GFP regulated by 12xlambda-TPL (LV-12xlambda-TPL-GFP). Transduction was first performed in 96 well plates and cells were induced with 100 μg/ml cumate (Sigma-Aldrich) and 10 nM coumermycin (Promega). Clones were selected for GFP intensity and a second screen was next performed for GFP expression under On/Off conditions (with and without inducers).

Comparison of induction capacity of the cumate and cumate/coumermycin switches.

Clones of 293SF-CymR, 293SF-CymR/rcTA and 293SF-CymR/XR-GryB were transduced with lentiviral vectors in the presence of 8 μg/ml of polybrene. 293SF-CymR and 293SF-CymR/rcTA were transduced with LV-CMV5CuO-GFP and with LV-CR5-GFP respectively at an MOI of 20 TU and the cells were induced by the addition of 100 μg/ml of cumate the next day. The 293SF-CymR/λR-GyrB clones were transduced with LV-12xlambda-TPL-GFP at an MOI of 5 and the cells were induced by addition of 100 μg/ml of cumate and 10 nM of coumermycin the next day. The cells were fixed and processed for flow cytometry analysis at 72 h post transduction.

### Packaging Cells for Lentiviral Vectors (293SF-PacLVIIIA)

293SF-CymR/λR-GyrB clone 7-2 was transfected in suspension using PEIpro® with pSB213 (p11xlambda-hbgmin-Gag/polIIIb), pVR19 (pVV-13xlambda-TPL-VSVg-Q96H-I57L), pVR21 (pVV-13xlambda-TPL-Rev) and pHygro at a DNA ratio of 40%, 25%, 25% and 10% respectively. Plasmids were digested with BspHI, ZraI, XmnI and XmnI, respectively. 36 h post-transfection, the cells were diluted at 0.35×10⁶ cells/ml with medium containing 65 μg/mL of hygromycin. After 4 and 8 days, cells were plated in nanowells (ALS Automated Lab Solutions GmbH, Jena, Germany) at 4.7 cells/nanowell with a hygromycin concentration of 50 μg/ml. Selection in suspension was continued in parallel. 205 (from 4 days selection in suspension) and 171 (from 8 days selection in suspension) resistant colonies were pooled together to form 2 distinct mini-pools. The mini-pools and the pool obtained in suspension were cloned by dilution into nanowell plates at a cell density of 0.6 cell/nanowell. Isolated cells in nanowells were documented at day 0 using the camera system of the CellCelector™ (Robotic cell picker). In total, 366 colonies were isolated using the CellCelector™ and transferred into a 384 well plate.

### Packaging Cells for Lentiviral Vectors (293SF-PacLVIIIB)

293SF-CymR/λR-GyrB clone 7-2 was transfected in suspension with PEIpro® using pSB211 (pCAG-Gag/polIIIb), pVR19 (pVV-13xlambda-TPL-VSVg-Q96H-I57L), pVR21 (pVV-13xlambda-TPL-Rev) and pHygro at a DNA ratio of 40%, 25%, 25% and 10% respectively. Plasmids were digested with BspHI, ZraI, XmnI and XmnI, respectively. At 36 h post-transfection, the cells were diluted at 0.5×10⁶ cells/ml with medium containing 80 μg/mL of hygromycin. After 8 days, the cells were plated in nanowell plates at 1.4 cells/nanowells with a hygromycin concentration of 50 or 25 μg/ml. 173 resistant colonies were pooled together to form a mini-pool. The mini-pool was cloned 6 days later by dilution into Nanowell plates at a cell density of 0.6 cell/nanowell in medium supplemented with 20 μg/ml hygromycin. Pictures demonstrating the presence of single cells in nanowells were obtained at day 0 using the camera system of the CellCelector™ (Robotic cell picker). 348 colonies were isolated using the CellCelector™ and transferred into a 384 well plate.

### Producer Cells for Lentiviral Vectors (293SF-LVPIIIB-GFP)

The packaging cells 293SF-PacLVIIIB, clone 3D4, was transfected in suspension using PEIpro® with the plasmids pNC111 (pNRC-LV1-CMVGFP) and pSB201 (pMPG/TKneo) at a DNA ratio of 4:1. Plasmids were previously digested with FspI and XbaI respectively. At 36 h post-transfection, the cells were diluted at 0.5×10⁶ cells/ml with medium containing 400 μg/mL of geneticin (Gibco). After 18 days in selection, cells were diluted for cloning in nanowells at a cell density of 0.6 cells/nanowell (no G418). Pictures demonstrating the presence of single cells in nanowells were obtained at day 0 using the camera system of the CellCelector™. 380 colonies were isolated using the CellCelector™ and transferred into a 384 well plate.

Screening of Clones from Packaging Cells (293 SF-PacLVIIIA, 293 SF-PacLVIIIB) and Producer Cells (293SF-LVPIIIB-GFP).

The clones were analyzed for the production of lentivirus expressing GFP (LV-CMV-GFP). They were first tested in 96 well plates, then in 24 well plates and finally in 6 well plate suspension. The clones from the packaging cells were transfected with pCSII-CMV-GFP (Broussau et al., 2008) and induced by adding cumate/coumermycin and sodium butyrate. The clones from the producers (293SF-LVPIIIB-GFP) were not transfected but were induced with cumate/coumermycin and sodium butyrate. The LV produced (LV-CMV-GFP) was titrated by transduction of 293A cells and GFP level was evaluated by fluorescence microscopy observation for LVs produced in 96- and 24-well plates and titrated by flow cytometry for LV produced in 6 well plates as described (Broussau et al., 2008).

### Western Blotting for Gag/Pol, VSVg and REV

Cells from 293SF-PacLVIIIB, clone 3D4 were tested by western blot for the expression of p24, VSVg and REV proteins. On the day of induction, 25 million cells were centrifuged and re-suspended in 25 ml of HyCell™ media to a final concentration of 1.0×10⁶ cells/mL in 125 ml shake flasks. Induction was done 1 h later using 80 μg/ml of cumate and 10 nM of coumermycin. As negative control, 5 million of 293SFCymR/λR-GyRB cells were centrifuged and transferred at −80° C. Two groups of cells were prepared with and without the addition of 8 mM of Sodium Butyrate at 18 h post induction. Five ml of cell cultures were harvested at 0, 24, 48, 72 h post induction. The cells were harvested by centrifugation and the cell pellet was transferred to −80° C. For western blot analysis, the cells were thawed and lysed with RIPA buffer (50 mM Tris-HCl pH 8, 150 mM NaCl, 0.1% SDS, 1% NP-40, 0.25% Na deoxycholate). After 30 min incubation on ice, the samples were sonicated and the lysates were clarified by centrifugation. Protein concentration was determined by BIO-RAD DC™ protein Assay (Bio-Rad Laboratories). The same amount of total protein was separated through a NuPAGE™ 4-12% Bis-Tris Gel, (Invitrogen) and analyzed by western blotting using Anti-HIV1 REV Mouse monoclonal antibody (ab85529, abcam), Rabbit polyclonal HIV p24 Ab (ProSci Catalog #7313), Rabbit polyclonal anti-VSVg tag antibody (ab83196, abcam), followed by a horseradish peroxidase-conjugated Donkey anti Rabbit immunoglobulin (Ig)G antibody or horseradish peroxidase-conjugated Sheep anti mouse immunoglobulin (Ig)G antibody (GE Healthcare UK Limited). The signal was revealed by chemiluminescence using the ECL™ Western Blotting Detection Reagents (Perkin Elmer, Inc) and analyzed with a digital imaging system (ImageQuant™ LAS 4000 mini biomolecular imager, GE Healthcare).

### Generation of 293SF-Rep Cells

A pool of cells expressing Rep 52, 68 and 78 was generated by transfecting 293SF-CymR/λR-GyrB with pVR43, pVR42, pVR41 and pUC57-TK-Hygro in a proportion of 55%, 30%, 15% and 10% respectively, using PEIpro® in Hycell™ medium supplemented with 4 mM glutamine and 0.1% Koliphore®. Before transfection, the Rep-encoding plasmids were linearized with SpeI and pUC57-TK-Hygro was linearized with XmnI. At 48 h post-transfection, the cells were centrifuged and resuspended in medium containing hygromycin (40 or 50 μg/ml) to give a final concentration of 0.5×10⁶ cells/ml. Viability and cell growth were monitored periodically. A small bank of frozen vials from the cells of the pool after about three weeks in culture were made.

One vial of the cell bank was thawed in Hycell™ medium supplemented with 4 mM glutamine and 0.1% Kolliphor® without selection. The selection hygromycin at 40 μg/mL was added two days after thawing. Cells were diluted three times with the selection before subcloning in 96 well plate. Subcloning was done in two media without selection: Hycell™ supplemented with 4 mM glutamine and 0.1% Kolliphor® and HSFM supplemented with 6 mM glutamine and 10 mg/l of transferrin 10 mg/L.

Colonies from the 96 wells were transferred to 24 well plates. When the wells became confluent, one third of the cell population was transferred into another 24 well plate to test for the production of AAV by transient transfection. For the transfection, the culture medium was replaced with fresh medium and the cells were transfected using 1 μg/ml of plasmid and 2 μg/ml of PEIpro®. Transfection was done using mixture of ptt3CAP1, pHelper (CellBiolab) and pAAV-GFP (CellBiolab). Induction was performed 4 h post transfection with 50 μg/ml of cumate and 10 nM of coumermycin. Harvest was performed 72 h post-transfection and the titration was performed using the standard gene transfer assay for AAV (described below) on 293SF-3F6 cells using BalanCD® medium. Clones that were able to produce AAV were amplified and vials of frozen cells were prepared.

### Western Blot Analysis of Rep Expression

Cells from 293SF-Rep clones (#13, 18, 35 and 36) were adapted in HyCell™ medium supplemented with 40 μg/ml Hygromycin. Clones were tested by western blot for the presence of REP protein without induction or after induction with different concentrations of cumate and coumermycin. 2.5 million cells were centrifuged and re-suspended in 2.5 ml of fresh HyCell™ media at a concentration of 1.0×10⁶ cells/mL in 6 well plate. The cells were induced with the following concentrations of cumate (in μg/ml) and coumermycin (in nM): 0.5 μg/ml/1 nM; 5 μg/ml/1 nM; or 50 μg/ml/10 nM. Cell cultures were harvested by centrifugation at 72 h post-induction and the cell pellets were transferred to −80° C. The cell pellets were thawed and lysed using 300 μl of RIPA (50 mM Tris-HCl pH 8, 150 mM NaCl, 0.1% SDS, 1% NP-40, 0.25% Na deoxycholate). After 30 min incubation on ice, the samples were sonicated and the lysates were clarified by centrifugation. Protein concentration was determined by BIO-RAD DC™ protein Assay (Bio-Rad Laboratories). The same amount of total protein (30 μg) was migrated through a NuPAGE™ 4-12% Bis-Tris Gel (Invitrogen) and analyzed by western blotting using Mouse Monoclonal IgG1 Anti-REP AAV (ARP American Research Products, Inc. catalog #03-61069), followed by a horseradish peroxidase-conjugated Sheep anti mouse immunoglobulin (Ig)G antibody (GE Healthcare UK Limited). The signal was revealed by chemiluminescence using the ECL™ western Blotting detection reagents (Perkin Elmer, Inc) and analyzed with a digital imaging system (ImageQuant™ LAS 4000 mini biomolecular imager, GE Healthcare).

### Production of AAV Using 29SF-Rep Cells and Gene Transfer Assay

293SF-Rep cells (clone 13) were centrifuged (300× g for 5 min.) to eliminate the Hygromycin (used to maintain the selection for the clone) and resuspended in Hycell™ TransFx-H (Hyclone) to have a final density of 1.0×10⁶ cells/mL in 6-well plate 2 h before transfection. The cell suspension was transfected with 1 μg/mL of DNA (using different ratios of pAAV-GFP and pHelper from Cell Biolabs, and pVR46-Cap, (FIG. 12)) and 2 μg/mL of PEIpro® (Polyplus) and incubated 4 h before adding the inducers. Plasmid mix with both inducers was prepared and added to the transfected cells to have a final concentration of 10 nM of coumermycin and 50 μg/mL of cumate. The induced cells were lysed after 3 days incubation with a lysis solution at a final concentration of 2 mM of MgCl2 (Sigma-Aldrich), 0.1% of Triton™ X-100 (Sigma-Aldrich) and 2.5 U/mL of Benzonase (MilliporeSigma). After 2 h of lysis, MgSO4 was added at a final concentration of 37.5 mM to stabilize the viral particles. The lysed cells were then centrifuged at 13 000 rpm for 3 minutes and the supernatant was harvested and frozen. The titer of AAV produced was measured using cells in BalanCD® HEK293 medium (FUJIFILM Irvine Scientific). 293SF-Rep cells were plated at 0.5×10⁶ cells/mL in 12-well plate and infected with a recombinant adenovirus vector encoding luciferase (HD-LUC, ΔE1, ΔE3) (Umana et al., 2001) at a multiplicity of infection (MOI) of 5. The cell lysate containing AAV was diluted 1:10 to 1:300 and added to the infected cells. After 24 h incubation, total cell density and viability were recorded and the 293SF-Rep cells were fixed with 2% Formaldehyde (Polysciences Inc.) and analyzed on flow cytometer (BD LSRFortessa™). The GFP % of single cells (10 000 events) was used to calculate the titer in infectious virus particles (IVP)/mL, considering the dilution factor.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

Table of Sequences
CymR gene (SEQ ID NO: 1) Pseudomonas putida
ATGAGCCCCAAGAGGAGAACCCAGGCCGAGAGAGCCATGGAGACCCAGGG
CAAGCTGATCGCCGCTGCCCTGGGCGTGCTGAGAGAGAAGGGCTACGCCG
GCTTCAGAATCGCCGACGTGCCTGGAGCCGCCGGAGTGAGCAGAGGCGCC
CAGAGCCACCACTTCCCTACCAAGCTGGAGCTGCTGCTGGCCACCTTCGA
GTGGCTGTACGAGCAGATCACCGAGAGGAGCAGAGCCAGACTGGCCAAGC
TGAAGCCCGAGGACGATGTGATCCAGCAGATGCTGGATGATGCCGCCGAG
TTCTTCCTGGACGACGACTTCAGCATCAGCCTGGACCTGATCGTGGCCGC
CGACAGAGACCCCGCCCTGAGAGAGGGCATCCAGAGGACCGTGGAGCGGA
ACAGATTCGTGGTGGAGGACATGTGGCTGGGAGTGCTGGTGTCCAGAGGC
CTGAGCAGAGATGACGCCGAGGACATCCTGTGGCTGATCTTCAACTCTGT
GAGGGGCCTGGCTGTGAGAAGCCTGTGGCAGAAGGACAAGGAGAGATTCG
AGAGAGTGCGGAACAGCACCCTGGAGATCGCCAGAGAGCGCTACGCCAAG
TTTAAACGGTGA
CymR protein (SEQ ID NO: 2) Pseudomonas putida
MSPKRRTQAERAMETQGKLIAAALGVLREKGYAGFRIADVPGAAGVSRGA
QSHHFPTKLELLLATFEWLYEQITERSRARLAKLKPEDDVIQQMLDDAAE
FFLDDDFSISLDLIVAADRDPALREGIQRTVERNRFVVEDMWLGVLVSRG
LSRDDAEDILWLIFNSVRGLAVRSLWQKDKERFERVRNSTLEIARERYAK
FKR*
CuO (P2) from CMV5-CuO (SEQ ID NO: 3)
Pseudomonas putida
AACAAACAGACAATCTGGTCTGTTTGTA
CuO (P1) (SEQ ID NO: 4) Pseudomonas putida
AGAAACAAACCAACCTGTCTGTATTA
CMV5-CuO (SEQ ID NO: 5) synthetic
CGTTACATAACTTACGGTAAATGGCCCGCCTGGCTGACCGCCCAACGACC
CCCGCCCATTGACGTCAATAATGACGTATGTTCCCATAGTAACGCCAATA
GGGACTTTCCATTGACGTCAATGGGTGGAGTATTTACGGTAAACTGCCCA
CTTGGCAGTACATCAAGTGTATCATATGCCAAGTCCGCCCCCTATTGACG
TCAATGACGGTAAATGGCCCGCCTGGCATTATGCCCAGTACATGACCTTA
CGGGACTTTCCTACTTGGCAGTACATCTACGTATTAGTCATCGCTATTAC
CATGGTGATGCGGTTTTGGCAGTACACCAATGGGCGTGGATAGCGGTTTG
ACTCACGGGGATTTCCAAGTCTCCACCCCATTGACGTCAATGGGAGTTTG
TTTTGGCACCAAAATCAACGGGACTTTCCAAAATGTCGTAATAACCCCGC
CCCGTTGACGCAAATGGGCAAGCTTGCCGGGTCGAGGTAGGCGTGTACGG
TGGGAGGCCTATATAAGCAACCGGTATAATACAAACAGACCAGATTGTCT
GTTTGTTACCGGTGTTTAGTGAACCGGGCGCGCCTCATATCGCCTGGAGA
CGCCATCCACGCTGTTTTGACCTCCATAGAAGACACCGGGACCGATCCAG
CCTCCGCGGTCACTCTCTTCCGCATCGCTGTCTGCGAGGGCCAGCTGTTG
GGCTCGCGGTTGAGGACAAACTCTTCGCGGTCTTTCCAGTACTCTTGGAT
CGGAAACCCGTCGGCCTCCGAACGGTACTCCGCCACCGAGGGACCTGAGC
CAGTCCGCATCGACCGGATCGGAAAACCTCTCGAGAAAGGCGTCTAACCA
GTCACAGTCGCAAGGTAGGCTGAGCACCGTGGCGGGCGGCAGCGGGTGGC
GGTCGGGGTTGTTTCTGGCGGAGGTGCTGCTGATGATGTAATTAAAGTAG
GCGGTCTTGAGCCGGCGGATGGTCGAGGTGAGGTGTGGCAGGCTTGAGAT
CCAGCTGTTGGGGTGAGTACTCCCTCTCAAAAGCGGGCATGACTTCTGCG
CTAAGATTGTCAGTTTCCAAAAACGAGGAGGATTTGATATTCACCTGGCC
C
1xlambdaOP (SEQ ID NO: 6) bacteriophage
TCGAGTTTACCTCTGGCGGTGATAG
12xlambdaOP (SEQ ID NO: 7) synthetic
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
13xlambdaOP (SEQ ID NO: 8) synthetic
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAG
12xlambdaCMVmin (SEQ ID NO: 9) synthetic
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGACTCTAGATAGGCGTGTACGGTGGGAGGCCTATATAAGCAGAGCT
13xlambda CMVmin (SEQ ID NO: 10) synthetic
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGACTCTAGATAGGCGTGTACGGT
GGGAGGCCTATATAAGCAGAGCT
13xlambda-TPL (SEQ ID NO: 11) synthetic
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGACTCTAGATAGGCGTGTACGGT
GGGAGGCCTATATAAGCAGAGCTCGTTTAGTGAACCGTCAGATCGCCTGG
AGACGCCATCCACGCTGTTTTGACCTCCATAGAAGACACCGGGACCGATC
CAGCCTCCGCGGTCACTCTCTTCCGCATCGCTGTCTGCGAGGGCCAGCTG
TTGGGCTCGCGGTTGAGGACAAACTCTTCGCGGTCTTTCCAGTACTCTTG
GATCGGAAACCCGTCGGCCTCCGAACGGTACTCCGCCACCGAGGGACCTG
AGCGAGTCCGCATCGACCGGATCGGAAAACCTCTCGAGAAAGGCGTCTAA
CCAGTCACAGTCGCAAGGTAGGCTGAGCACCGTGGCGGGCGGCAGCGGGT
GGCGGTCGGGGTTGTTTCTGGCGGAGGTGCTGCTGATGATGTAATTAAAG
TAGGCGGTCTTGAGACGGCGGATGGTCGAGGTGAGGTGTGGCAGGCTTGA
GATCCAGCTGTTGGGGTGAGTACTCCCTCTCAAAAGCGGGCATTACTTCT
GCGCTAAGATTGTCAGTTTCCAAAAACGAGGAGGATTTGATATTCACCTG
GCCC
11xlambda-hbgmin (SEQ ID NO: 12) synthetic
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGAGTTTACCTCTGGCGGTGATAG
TCGAGTTTACCTCTGGCGGTGATAGTCGACTCTAGATAGGCGTGTACGGT
GGGAGGCCTATATAAGCAGAGCTCGTTTAGTGAACCGTCAGATCCCTGGA
GACGCCATCCACGCTGTTTTGACCTCCATAGAAGACACCGGGACCGATCA
ACCTAAGCTTCCAACCGGTGTTTAGTGAACCGGGCGCGCCTCATATCGCC
TGGAGACGCCATCCACGCTGTTTTGACCTCCATAGAAGACACCGGGACCG
ATCCAGCCTCCGCGGTCACTCTCTTCCGCATCGCTGTCTGCGAGGGCCAG
CTGTTGGGCTCGCGGTTGAGGACAAACTCTTCGCGGTCTTTCCAGTACTC
TTGGATCGGAAACCCGTCGGCCTCCGAACGGTACTCCGCCACCGAGGGAC
CTGAGCGAGTCCGCATCGACCGGATCGGAAAACCTCTCGAGAAAGGCGTC
TAACCAGTCACAGTCGCAAGGTAGGCTGAGCACCGTGGCGGGCGGCAGCG
GGTGGCGGTCGGGGTTGTTTCTGGCGGAGGTGCTGCTGATGATGTAATTA
AAGTAGGCGGTCTTGAGACGGCGGATGGTCGAGGTGAGGTGTGGCAGGCT
TGAGATCCAGCTGTTGGGGTGAGTACTCCCTCTCAAAAGCGGGCATTACT
TCTGCGCTAAGATTGTCAGTTTCCAAAAACGAGGAGGATTTGATATTCAC
CTGGCCCGATCTGGCCATACACTTAACGTACACATATTGACCAAATCAGG
GTAATTTTGCATTTGTAATTTTAAAAAATGCTTTCTTCTTTTAATATACT
TTTTTGTTTATCTTATTTCTAATACTTTCCCTAATCTCTTTCTTTCAGGG
CAATAATGATACAATGTATCATGCCTCTTTGCACCATTCTAAAGAATAAC
AGTGATAATTTCTGGGTTAAGGCAATAGCAATATTTCTGCATATAAATAT
TTCTGCATATAAATTGTAACTGATGTAAGAGGTTTCATATTGCTAATAGC
AGCTACAATCCAGCTACCATTCTGCTTTTATTTTATGGTTGGGATAAGGC
TGGATTATTCTGAGTCCAAGCTAGGCCCTTTTGCTAATCATGTTCATACC
TCTTATCTTCCTCCCACAGCTC
λR-GyrB gene (SEQ ID NO: 13) synthetic
ATGAGCACAAAAAAGAAACCATTAACACAAGAGCAGCTTGAGGACGCACG
TCGCCTTAAAGCAATTTATGAAAAAAAGAAAAATGAACTTGGCTTATCCC
AGGAATCTGTCGCAGACAAGATGGGGATGGGGCAGTCAGGCGTTGGTGCT
TTATTTAATGGCATCAATGCATTAAATGCTTATAACGCCGCATTGCTTGC
AAAAATTCTCAAAGTTAGCGTTGAAGAATTTAGCCCTTCAATCGCCAGAG
AAATCTACGAGATGTATGAAGCGGTTGGGATGCAGCCGTCACTTAGAAGT
GAGTATGAGTACCCTGTTTTTTCTCATGTTCAGGCAGGGATGTTCTCACC
TGAGCTTAGAACCTTTACCAAAGGTGATGCGGAGAGATGGGTAGATATCT
CGAATTCTTATGACTCCTCCAGTATCAAAGTCCTGAAAGGGCTGGATGCG
GTGCGTAAGCGCCCGGGTATGTATATCGGCGACACGGATGACGGCACCGG
TCTGCACCACATGGTATTCGAGGTGGTAGATAACGCTATCGACGAAGCGC
TCGCGGGTCACTGTAAAGAAATTATCGTCACCATTCACGCCGATAACTCT
GTCTCTGTACAGGATGACGGGCGCGGCATTCCGACCGGTATTCACCCGGA
AGAGGGCGTATCGGCGGCGGAAGTGATCATGACCGTTCTGCACGCAGGCG
GTAAATTTGACGATAACTCCTATAAAGTGTCCGGCGGTCTGCACGGCGTT
GGTGTTTCGGTAGTAAACGCCCTGTCGCAAAAACTGGAGCTGGTTATCCA
GCGCGAGGGTAAAATTCACCGTCAGATCTACGAACACGGTGTACCGCAGG
CCCCGCTGGCGGTTACCGGCGAGACTGAAAAAACCGGCACCATGGTGCGT
TTCTGGCCCAGCCTCGAAACCTTCACCAATGTGACCGAGTTCGAATATGA
AATTCTGGCGAAACGTCTGCGTGAGTTGTCGTTCCTCAACTCCGGCGTTT
CCATTCGTCTGCGCGACAAGCGCGACGGCAAAGAAGACCACTTCCACTAT
GAAGGCGGCCCATGGATGGGCCCTAAAAAGAAGCGTAAAGTCGCCATCGA
TCAGCTCACCATGGTGTTTCCTTCTGGGCAGATCTCAAACCAGGCCCTGG
CCTTAGCACCGTCCTCTGCCCCAGTCCTTGCCCAGACCATGGTCCCTTCC
TCAGCCATGGTACCTCTGGCTCAGCCCCCAGCTCCTGCCCCAGTTCTAAC
CCCGGGTCCTCCCCAGTCCCTGTCTGCACCTGTTCCAAAGAGCACCCAGG
CTGGGGAAGGCACGCTGTCGGAAGCCCTGCTGCACCTGCAGTTTGATGCT
GATGAAGACTTGGGGGCCTTGCTTGGCAACAGCACAGACCCAGGAGTGTT
CACAGACCTGGCATCTGTGGACAACTCAGAGTTTCAGCAGCTCCTGAACC
AGGGTGTGTCCATGTCTCACTCCACAGCTGAGCCCATGCTGATGGAGTAC
CCTGAAGCTATAACTCGCCTGGTGACAGGGTCCCAGAGGCCCCCTGACCC
AGCTCCCACACCCCTGGGGACCTCGGGGCTTCCCAATGGTCTCTCCGGAG
ATGAAGACTTCTCCTCCATTGCGGACATGGACTTCTCTGCTCTGCTGAGT
CAGATCAGCTCCAGCGGCCAATAA
λR-GyrB protein (SEQ ID NO: 14) synthetic
MSTKKKPLTQEQLEDARRLKAIYEKKKNELGLSQESVADKMGMGQSGVGA
LENGINALNAYNAALLAKILKVSVEEFSPSIAREIYEMYEAVGMQPSLRS
EYEYPVFSHVQAGMFSPELRTFTKGDAERWVDISNSYDSSSIKVLKGLDA
VRKRPGMYIGDTDDGTGLHHMVFEVVDNAIDEALAGHCKEIIVTIHADNS
VSVQDDGRGIPTGIHPEEGVSAAEVIMTVLHAGGKFDDNSYKVSGGLHGV
GVSVVNALSQKLELVIQREGKIHRQIYEHGVPQAPLAVTGETEKTGTMVR
FWPSLETFTNVTEFEYEILAKRLRELSFLNSGVSIRLRDKRDGKEDHFHY
EGGPWMGPKKKRKVAIDQLTMVFPSGQISNQALALAPSSAPVLAQTMVPS
SAMVPLAQPPAPAPVLTPGPPQSLSAPVPKSTQAGEGTLSEALLHLQFDA
DEDLGALLGNSTDPGVFTDLASVDNSEFQQLLNQGVSMSHSTAEPMLMEY
PEAITRLVTGSQRPPDPAPTPLGTSGLPNGLSGDEDFSSIADMDFSALLS
QISSSGQ
C-terminal portion of the p65 subunit of mouse
NF-κB (SEQ ID NO: 15)
PSGQISNQALALAPSSAPVLAQTMVPSSAMVPLAQPPAPAPVLTPGPPQS
LSAPVPKSTQAGEGTLSEALLHLQFDADEDLGALLGNSTDPGVFTDLASV
DNSEFQQLLNQGVSMSHSTAEPMLMEYPEAITRLVTGSQRPPDPAPTPLG
TSGLPNGLSGDEDFSSIADMDFSALLSQISS
Rabbit β-globin polyA (SEQ ID NO: 16)
AATAAAGGAAATTTATTTTCATTGCAATAGTGTGTTGGAATTTTTTGTGT
CTCTCA
bGH (bovine growth hormone) polyA (SEQ ID NO: 17)
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGATGCGGTGGGCTCTATGG
SV40 poly(A) signal (SEQ ID NO: 18) simian
virus 40
AACTTGTTTATTGCAGCTTATAATGGTTACAAATAAAGCAATAGCATCAC
AAATTTCACAAATAAAGCATTTTTTTCACTGCATTCTAGTTGTGGTTTGT
CCAAACTCATCAATGTATCTTA
Codon optimized VSVg-Q96-157L (SEQ ID NO: 19)
synthetic
ATGAAATGTCTGCTGTACCTGGCATTCCTGTTTATCGGAGTCAACTGCAA
GTTTACTATCGTCTTCCCCCACAATCAGAAAGGCAATTGGAAGAACGTGC
CAAGCAATTACCACTATTGCCCCAGCTCCTCTGACCTGAACTGGCATAAT
GATCTGATCGGCACCGCCCTGCAGGTCAAGATGCCCAAATCCCACAAGGC
CATCCAGGCTGACGGGTGGATGTGCCATGCTTCTAAATGGGTGACCACAT
GTGACTTCCGGTGGTACGGACCAAAGTATATCACTCATAGCATTCGCTCC
TTCACCCCCTCCGTGGAGCAGTGCAAAGAGTCTATTGAACAGACCAAGCA
GGGGACATGGCTGAACCCTGGATTTCCCCCTCAGTCCTGTGGGTACGCCA
CAGTCACTGACGCTGAGGCAGTGATCGTCCAGGTGACACCACACCATGTC
CTGGTGGACGAGTATACTGGGGAATGGGTGGATTCACAGTTCATTAACGG
AAAATGCAGCAATTACATCTGTCCTACAGTCCACAACTCTACTACCTGGC
ATAGTGATTATAAGGTGAAAGGCCTGTGCGATAGCAATCTGATCTCCATG
GACATTACTTTCTTTAGTGAGGATGGCGAACTGAGTTCACTGGGGAAGGA
GGGAACCGGCTTTCGGAGCAATTACTTCGCATATGAAACAGGCGGGAAAG
CCTGCAAGATGCAGTACTGTAAACACTGGGGAGTCCGCCTGCCATCTGGC
GTGTGGTTCGAGATGGCAGACAAGGATCTGTTTGCCGCTGCACGATTCCC
AGAGTGCCCCGAAGGCAGCTCCATCTCTGCCCCCAGTCAGACTTCAGTGG
ACGTGAGCCTGATTCAGGATGTGGAGAGAATCCTGGACTACAGTCTGTGC
CAGGAAACCTGGTCAAAAATTAGGGCTGGCCTGCCTATCTCACCAGTGGA
CCTGAGCTATCTGGCTCCCAAAAACCCTGGGACTGGACCCGCCTTCACCA
TCATTAATGGGACACTGAAGTACTTCGAGACCCGGTATATCAGAGTGGAC
ATTGCCGCTCCTATCCTGAGCCGAATGGTGGGCATGATCTCCGGGACAAC
TACCGAGCGGGAACTGTGGGACGATTGGGCTCCTTACGAGGATGTCGAAA
TTGGACCAAACGGCGTGCTGAGGACATCTAGTGGCTACAAATTTCCTCTG
TATATGATCGGCCACGGGATGCTGGACTCTGATCTGCATCTGTCAAGCAA
GGCACAGGTGTTCGAGCACCCCCATATCCAGGACGCAGCCTCTCAGCTGC
CTGACGATGAAAGTCTGTTCTTTGGGGATACCGGACTGAGCAAAAATCCA
ATTGAGCTGGTGGAAGGATGGTTTTCCTCTTGGAAGAGTTCAATCGCCTC
CTTCTTTTTCATCATTGGACTGATCATTGGCCTGTTCCTGGTCCTGCGGG
TGGGCATTCACCTGTGCATCAAGCTGAAACATACCAAGAAAAGACAGATT
TACACCGACATTGAGATGAACAGACTGGGCAAGTGA
VSVg-Q96-157L amino acid (SEQ ID NO: 20) vesicular
stomatitis virus
MKCLLYLAFLFIGVNCKFTIVFPHNQKGNWKNVPSNYHYCPSSSDLNWHN
DLIGTALQVKMPKSHKAIQADGWMCHASKWVTTCDFRWYGPKYITHSIRS
FTPSVEQCKESIEQTKQGTWLNPGFPPQSCGYATVIDAEAVIVQVTPHHV
LVDEYTGEWVDSQFINGKCSNYICPTVHNSTTWHSDYKVKGLCDSNLISM
DITFFSEDGELSSLGKEGTGFRSNYFAYETGGKACKMQYCKHWGVRLPSG
VWFEMADKDLFAAARFPECPEGSSISAPSQTSVDVSLIQDVERILDYSLC
QETWSKIRAGLPISPVDLSYLAPKNPGTGPAFTIINGTLKYFETRYIRVD
IAAPILSRMVGMISGTTTERELWDDWAPYEDVEIGPNGVLRTSSGYKFPL
YMIGHGMLDSDLHLSSKAQVFEHPHIQDAASQLPDDESLFFGDTGLSKNP
IELVEGWFSSWKSSIASFFFIIGLIIGLFLVLRVGIHLCIKLKHTKKRQI
YTDIEMNRLGK*

### REFERENCES

Balakrishnan, B., and G. R. Jayandharan. 2014. Basic biology of adeno-associated virus (AAV) vectors used in gene therapy. Curr. Gene Ther. 14:86-100.
Blain, M., Y. Zeng, M. Bendjelloul, P. L. Hallauer, A. Kumar, K. E. Hastings, G. Karpati, B. Massie, and R. Gilbert. 2010. Strong muscle-specific regulatory cassettes based on multiple copies of the human slow troponin I gene upstream enhancer. Hum. Gene Ther. 21:127-134.
Broussau, S., N. Jabbour, G. Lachapelle, Y. Durocher, R. Tom, J. Transfiguracion, R. Gilbert, and B. Massie. 2008. Inducible Packaging Cells for Large-scale Production of Lentiviral Vectors in Serum-free Suspension Culture. Mol. Ther. 16:500-507.
Caron, A. W., C. Nicolas, B. Gaillet, I. Ba, M. Pinard, A. Gamier, B. Massie, and R. Gilbert. 2009. Fluorescent labeling in semi-solid medium for selection of mammalian cells secreting high-levels of recombinant proteins. BMC. Biotechnol. 9:42.
Chahal, P., S. M. Elahi, P. O'Neal, M. H. Venne, N. Nazemi-Moghaddam, and R. Gilbert. 2018. Key Rep-proteins necessary for the adeno-associated virus production by transient transfection in HEK293 cells in suspension and serum-free medium. In 21th Annual Meeting, American Society of Gene and Cell Therapy, May 16-19, 2018, Chicago, IL.
Cockrell, A. S., and T. Kafri. 2007. Gene delivery by lentivirus vectors. Mol Biotechnol. 36:184-204.
Cote, J., A. Gamier, B. Massie, and A. Kamen. 1998. Serum-free production of recombinant proteins and adenoviral vectors by 293SF-3F6 cells. Biotechnol. Bioeng. 59:567-575.
Dropulic, B. 2011. Lentiviral vectors: their molecular design, safety, and use in laboratory and preclinical research. Hum. Gene Ther. 22:649-657.
Durocher, Y., S. Perret, and A. Kamen. 2002. High-level and high-throughput recombinant protein production by transient transfection of suspension-growing human 293-EBNA1 cells. Nucleic Acids Res. 30:E9.
Escors, D., and K. Breckpot. 2010. Lentiviral vectors in gene therapy: their current status and future potential. Arch. Immunol. Ther. Exp. (Warsz). 58:107-119.
Farson, D., R. Witt, R. McGuinness, T. Dull, M. Kelly, J. Song, R. Radeke, A. Bukovsky, A. Consiglio, and L. Naldini. 2001. A new-generation stable inducible packaging cell line for lentiviral vectors. Hum. Gene Ther. 12:981-997.
Gervais, C., D. Paquette, A. Burns-Tardis, L. Martin, and B. Massie. 1998. Development of high output expression vectors for antibody production in mammalian cells. In Animal Cell Technology: Basic & Applied Aspects. K. Nagai and M. Wachi, editors. Kluwer Academic Publishers, Netherlands. 349-354.
Gilbert, R., S. Broussau, and B. Massie. 2007. Protein production using lentiviral vectors. In Methods Express: expression systems. M. R. Dyson and Y. Durocher, editors. Scion Publishing Ltd, Oxfordshire. 241-258.
Gilbert, R., C. Guilbault, D. Gagnon, A. Bernier, L. Bourget, S. M. Elahi, A. Kamen, and B. Massie. 2014. Establishment and validation of new complementing cells for production of E1-deleted adenovirus vectors in serum-free suspension culture. J. Virol. Methods. 208:177-188.
Gossen, M., and H. Bujard. 1992. Tight control of gene expression in mammalian cells by tetracycline-responsive promoters. Proc. Natd. Acad. Sci. U.S.A 89:5547-5551.
Grieger, J. C., and R. J. Samulski. 2012. Adeno-associated virus vectorology, manufacturing, and clinical applications. Methods Enzymol. 507:229-254.
Kafri, T., P. H. van, L. Ouyang, F. H. Gage, and I. M. Verma. 1999. A packaging cell line for lentivirus vectors. J. Virol. 73:576-584.
Kotterman, M. A., and D. V. Schaffer. 2014. Engineering adeno-associated viruses for clinical gene therapy. Nat. Rev. Genet. 15:445-451.
Massie, B., F. Couture, L. Lamoureux, D. D. Mosser, C. Guilbault, P. Jolicoeur, F. Belanger, and Y. Langelier. 1998a. Inducible overexpression of a toxic protein by an adenovirus vector with a tetracycline-regulatable expression cassette. J Virol. 72:2289-2296.
Massie, B., D. D. Mosser, M. Koutroumanis, I. Vitte-Mony, L. Lamoureux, F. Couture, L. Paquet, C. Guilbault, J. Dionne, D. Chahla, P. Jolicoeur, and Y. Langelier. 1998b. New adenovirus vectors for protein production and gene transfer. Cytotechnology. 28:53-64.
Matrai, J., M. K. Chuah, and T. VandenDriessche. 2010. Recent advances in lentiviral vector development and applications. Mol. Ther. 18:477-490.
Mercille, S., P. Jolicoeur, C. Gervais, D. Paquette, D. D. Mosser, and B. Massie. 1999. Dose-dependent reduction of apoptosis in nutrient-limited cultures of NS/0 myeloma cells transfected with the E1B-19K adenoviral gene. Biotechnol. Bioeng. 63:516-528.
Milone, M. C., and U. O'Doherty. 2018. Clinical use of lentiviral vectors. Leukemia. 32:1529-1541.
Miyazaki, J., S. Takaki, K. Araki, F. Tashiro, A. Tominaga, K. Takatsu, and K. Yamamura. 1989. Expression vector system based on the chicken beta-actin promoter directs efficient production of interleukin-5. Gene. 79:269-277.
Miyoshi, H., U. Blomer, M. Takahashi, F. H. Gage, and I. M. Verma. 1998. Development of a self-inactivating lentivirus vector. J. Virol. 72:8150-8157.
Mullick, A., Y. Xu, R. Warren, M. Koutroumanis, C. Guilbault, S. Broussau, F. Malenfant, L. Bourget, L. Lamoureux, R. Lo, A. W. Caron, A. Pilotte, and B. Massie. 2006. The cumate gene-switch: a system for regulated expression in mammalian cells. BMC Biotechnol. 6:43.
Ni, Y., S. Sun, I. Oparaocha, L. Humeau, B. Davis, R. Cohen, G. Binder, Y. N. Chang, V. Slepushkin, and B. Dropulic. 2005. Generation of a packaging cell line for prolonged large-scale production of high-titer HIV-1-based lentiviral vector. J. Gene Med. 7:818-834.
Pacchia, A. L., M. E. Adelson, M. Kaul, Y. Ron, and J. P. Dougherty. 2001. An inducible packaging cell system for safe, efficient lentiviral vector production in the absence of HIV-1 accessory proteins. Virology. 282:77-86.
Pluta, K., and M. M. Kacprzak. 2009. Use of HIV as a gene transfer vector. Acta Biochim. Pol. 56:531-595.
Poulain, A., S. Perret, F. Malenfant, A. Mullick, B. Massie, and Y. Durocher. 2017. Rapid protein production from stable CHO cell pools using plasmid vector and the cumate gene-switch. J Biotechnol. 255:16-27.
Robert, M. A., P. S. Chahal, A. Audy, A. Kamen, R. Gilbert, and B. Gaillet. 2017. Manufacturing of recombinant adeno-associated viruses using mammalian expression platforms. Biotechnol. J. 12.
Sanber, K. S., S. B. Knight, S. L. Stephen, R. Bailey, D. Escors, J. Minshull, G. Santilli, A. J. Thrasher, M. K. Collins, and Y. Takeuchi. 2015. Construction of stable packaging cell lines for clinical lentiviral vector production. Sci Rep. 5:9021.
Sparacio, S., T. Pfeiffer, H. Schaal, and V. Bosch. 2001. Generation of a flexible cell line with regulatable, high-level expression of HIV Gag/Pol particles capable of packaging HIV-derived vectors. Mol. Ther. 3:602-612.
Umana, P., C. A. Gerdes, D. Stone, J. R. Davis, D. Ward, M. G. Castro, and P. R. Lowenstein. 2001. Efficient FLPe recombinase enables scalable production of helper-dependent adenoviral vectors with negligible helper-virus contamination. Nat. Biotechnol. 19:582-585.
Vigna, E., S. Cavalieri, L. Ailles, M. Geuna, R. Loew, H. Bujard, and L. Naldini. 2002. Robust and efficient regulation of transgene expression in vivo by improved tetracycline-dependent lentiviral vectors. Mol. Ther. 5:252-261.
Weitzman, M. D., and R. M. Linden. 2011. Adeno-associated virus biology. Methods Mol. Biol. 807:1-23.
Wright, J. F. 2009. Transient transfection methods for clinical adeno-associated viral vector production. Hum. Gene Ther. 20:698-706.
Zhao, H. F., J. Boyd, N. Jolicoeur, and S. H. Shen. 2003. A coumermycin/novobiocin-regulated gene expression system. Hum. Gene Ther. 14:1619-1629.

1. An inducible expression system comprising:
a) a first expression cassette comprising a nucleic acid molecule encoding a cumate repressor protein operably linked to a constitutive promoter and a polyadenylation signal;
b) a second expression cassette comprising a nucleic acid molecule encoding a coumermycin chimeric transactivator protein operably linked to a cumate-inducible promoter and a polyadenylation signal; and
c) a third expression cassette comprising:
i) a nucleic acid molecule comprising a coumermycin-inducible promoter, a cloning site, and a polyadenylation signal, wherein the cloning site is for insertion of a nucleic acid molecule encoding a first RNA or protein of interest in operable linkage with the coumermycin-inducible promoter and the polyadenylation signal, or
ii) a nucleic acid molecule encoding a first RNA or protein of interest operably linked to a coumermycin-inducible promoter and a polyadenylation signal.

2. The expression system of claim 1, wherein the constitutive promoter is selected from the group consisting of human Ubiquitin C (UBC) promoter, human Elongation Factor 1alpha (EF1A) promoter, human phosphoglycerate kinase 1 (PGK) promoter, simian virus 40 early promoter (SV40), beta-actin promoter, cytomegalovirus immediate-early promoter (CMV), hybrid CMV enhancer/beta-actin promoter (CAG), and variants thereof.

3-6. (canceled)

7. The expression system of claim 1, wherein the coumermycin-inducible promoter further comprises a tripartite leader (TPL) and/or a major late promoter (MLP) enhancer.

8. The expression system of claim 7, wherein the coumermycin-inducible promoter comprises the nucleotide sequence set forth in SEQ ID NO: 11 or a functional variant thereof.

9-10. (canceled)

11. The expression system of claim 1, wherein the third expression cassette comprises the nucleic acid molecule encoding the first RNA or protein of interest operably linked to the coumermycin-inducible promoter and the polyadenylation signal.

12. (canceled)

13. The expression system of claim 1, further comprising a fourth expression cassette comprising a nucleic acid molecule encoding a second RNA or protein of interest operably linked to a promoter and a polyadenylation signal.

14. The expression system of claim 13, further comprising a fifth expression cassette comprising a nucleic acid molecule encoding a third RNA or protein of interest operably linked to a promoter and a polyadenylation signal.

15. The expression system of claim 13, wherein the promoter of the fourth and/or fifth expression cassette is a coumermycin-inducible promoter.

16. (canceled)

17. The expression system of claim 11, wherein the expression system encodes one or more components of a viral vector.

18. The expression system of claim 14, wherein:
the third expression cassette encodes lentiviral REV protein, the promoter of the fourth expression cassette is a coumermycin-inducible promoter and the fourth expression cassette encodes a viral envelope protein, and the fifth expression cassette encodes a lentiviral Gag/pol; or
wherein the third expression cassette encodes a viral envelope protein, the promoter of the fourth expression cassette is a coumermycin-inducible promoter and the fourth expression cassette encodes a lentiviral Gag/pol, and the fifth expression cassette encodes a lentiviral REV protein.

19. (canceled)

20. The expression system of claim 18, wherein the viral envelope protein is VSVg, optionally VSVg-Q96H-I57L.

21. The expression system of claim 13, wherein the third expression cassette encodes Rep 40 or Rep 52, the fourth expression cassette encodes Rep 68 or Rep 78, and the fourth expression cassette is under the control of a coumermycin-inducible promoter.

22-31. (canceled)

32. A cell comprising the expression system of claim 1.

33. The cell of claim 32, wherein the cell is a human cell.

34. The cell of claim 32, wherein the cell is a Human Embryonic Kidney (HEK)-293 cell or a derivative thereof, a Chinese Hamster Ovary (CHO) cell or a derivative thereof, a VERO cell or a derivative thereof, a HeLa cell or a derivative thereof, an A549 cell or a derivative thereof, a stem cell or a derivative thereof, or a neuron or a derivative thereof.

35. A method of producing an RNA or protein of interest, the method comprising culturing the cell of claim 32 in the presence of a cumate effector molecule and a coumermycin effector molecule, wherein the third expression cassette encodes the RNA or protein of interest and wherein the RNA or protein of interest is produced.

36. The method of claim 35, wherein the cumate effector molecule is cumate present at a concentration of about 1 to about 200 μg/ml and wherein the coumermycin effector molecule is coumermycin present at a concentration of about 1 to about 30 nM.

37. (canceled)

38. The method of claim 35, wherein the cell is grown in suspension and/or in the absence of serum.

39. A viral packaging cell comprising the expression system of claim 17.

40-41. (canceled)

42. The viral packaging cell of claim 39, further comprising a viral construct carrying a gene of interest.

43. A method of producing a viral vector, the method comprising:
a) obtaining the viral packaging cell of claim 42; and
b) culturing the cell in the presence of a cumate effector molecule and a coumermycin effector molecule, thereby producing the viral vector.

44-62. (canceled)
